(19) United States
(12) Patent Application Publication
(10) Pub. No.: US 2022/0154189 A1
(43) Pub. Date: May 19, 2022

(54) COMPOSITIONS AND METHODS FOR THE TREATMENT OF KRAS ASSOCIATED DISEASES OR DISORDERS

(71) Applicant: Dicerna Pharmaceuticals, Inc., Lexington, MA (US)

(72) Inventor: Shanthi GANESH, Shrewsbury, MA (US)

(21) Appl. No.: 17/442,301

(22) PCT Filed: Mar. 27, 2020
(86) PCT No.: PCT/US2020/025125

§ 371 Date: Sep. 23, 2021

Related U.S. Application Data
(60) Provisional application No. 62/826,121, filed on Mar. 29, 2019.

(51) Int. Cl. C12N 15/113; A61K 31/519; A61K 31/713; C07K 16/28; A61K 39/395; A61P 35/00

(52) U.S. Cl. CPC C12N 15/1137; A61K 31/519; A61K 31/713; C07K 16/2818; C12N 2320/31; A61K 39/3955; A61P 35/00; C12N 2310/14; C07K 16/2827

(57) ABSTRACT

Provided herein are methods of treating a KRAS-associated cancer in a subject, comprising administering to the subject a therapeutically-effective amount of a KRAS nucleic acid inhibitor molecule and a therapeutically-effective amount of an MEK inhibitor or an immunotherapeutic agent. Also disclosed herein is a method of potentiating a therapeutic effect of an immunotherapeutic agent against a KRAS-associated cancer, comprising administering to a subject having the KRAS-associated cancer a KRAS nucleic acid inhibitor molecule in an amount sufficient to potentiate the therapeutic effect of the immunotherapeutic agent against the cancer.

### CROSS REFERENCE TO RELATED APPLICATIONS

This application claims the benefit of, and relies on the filing date of, U.S. provisional patent application No. 62/826,121, filed 29 Mar. 2019. The entire contents of each related application referenced in this paragraph is incorporated herein by reference in its entirety.

### SEQUENCE LISTING

The instant application contains a Sequence Listing which has been filed electronically in ASCII format and is hereby incorporated by reference in its entirety. Said ASCII copy, created on Mar. 26, 2020 is named 0243_0034-PCT_SL.txt and is 15,508 bytes in size.

### FIELD

The present disclosure relates generally to combination therapy using a nucleic acid inhibitor molecule that reduces expression of the KRAS gene in combination with at least one immunotherapeutic agent or an MEK inhibitor, as well as to methods of potentiating a therapeutic effect of an immunotherapeutic agent using a KRAS nucleic acid inhibitor molecule.

### BACKGROUND

Ras is a family of genes involved in cell signaling pathways that control cell growth and cell death. Disregulated Ras signaling can lead to tumor growth and metastasis (Goodsell D. S. Oncologist 4:263-4). It is estimated that 20-25% of all human tumors contain activating mutations in Ras; in specific tumor types, such as pancreatic carcinomas, this figure can be as high as 90% (Downward J. Nat Rev Cancer, 3:11-22). Accordingly, members of the Ras gene family are attractive molecular targets for cancer therapeutic drugs.

The three human RAS genes encode highly-related 188 to 189 amino acid proteins, designated H-Ras, N-Ras, and K-Ras4A (KRAS isoform a) and K-Ras4B (KRAS isoform b; the two KRas proteins arise from alternative gene splicing). Ras proteins function as binary molecular switches that control intracellular signaling networks. Ras-regulated signal pathways control such processes as actin cytoskeletal integrity, proliferation, differentiation, cell adhesion, apoptosis, and cell migration. Ras and Ras-related proteins are often deregulated in cancers, leading to increased invasion and metastasis, and decreased apoptosis. Ras activates a number of pathways but an especially important one for tumorigenesis appears to be the mitogen-activated protein (MAP) kinases, which themselves transmit signals downstream to other protein kinases and gene regulatory proteins (Lodish et al. Molecular Cell Biology (4th ed.). San Francisco: W.H. Freeman, Chapter 25, “Cancer”). Accordingly, inhibiting KRAS gene expression can be used as a chemotherapeutic tool.

Double-stranded RNA (dsRNA) agents possessing strand lengths of 25 to 35 nucleotides have been described as effective inhibitors of target gene expression in mammalian cells (Rossi et al., U.S. Patent Application Nos. 2005/0244858 and US 2005/0277610), including KRAS gene expression (Brown, U.S. Pat. Nos. 9,200,284 and 9,809,819). dsRNA agents of such length are believed to be processed by the Dicer enzyme of the RNA interference (RNAi) pathway, leading such agents to be termed “Dicer substrate siRNA” (“DsiRNA”) agents. Additional modified structures of DsiRNA agents were previously described (Rossi et al., U.S. Patent Application No. 2007/0265220).

In certain instances, the immune system may also be involved in cancer treatment. The immune system uses certain molecules on the surface of immune cells as checkpoints to control T cell activation and prevent the immune system from targeting healthy cells and inducing autoimmunity. Certain cancer cells are able to take advantage of these immune checkpoint molecules to evade the immune system. In recent years, immunotherapeutic strategies to block immune checkpoint molecules, such as cytotoxic T-lymphocyte-associated protein-4 (CTLA-4) and programmed cell death receptor 1 (PD-1), have shown success against certain cancers. An anti-CTLA-4 monoclonal antibody (ipilimumab) was approved for the treatment of patients with advanced melanoma in 2011. An anti-PD-1 monoclonal antibody (nivolumab) was approved for the treatment of patients with certain advanced cancers in 2014, alone or in combination with ipilimumab. Other PD-1 inhibitors include, for example, prembrolizumab (Keytruda®) and nivolumab (Opdivo®). Antibodies that block immune checkpoint molecules like CTLA-4, PD-1, and PD-L1 appear to release the brakes on T cell activation and promote potent anti-tumor immune responses. However, only a subset of patients respond to this immunotherapy.

At least in certain instances, the tumors that respond to immunotherapy have a pre-existing T cell inflamed phenotype, with infiltrating T cells, a broad chemokine profile that recruits T cells to the tumor microenvironment, and high levels of IFN gamma secretion (also called hot or inflamed tumors). Gajewski et al., Nat Immunol., 2013, 14(10):1014-22; Ji et al., Cancer Immunol Immunother, 2012, 61:1019-31. Conversely, certain tumors that do not respond to immunotherapy have been shown to not have a T cell inflamed phenotype (also known as cold or non-inflamed tumors). Id.

There remains a need in the art to develop new cancer treatment options, including options that would make non-inflamed tumors responsive to immunotherapy.

### SUMMARY

This application is directed to methods of treatment comprising administering a KRAS nucleic acid inhibitor molecule and an immunotherapeutic agent or an MEK inhibitor to a subject. The KRAS nucleic acid molecules disclosed herein are capable of reducing the expression of KRAS mRNA in a cell, either in vitro or in a mammalian subject.

Disclosed herein is a method of treating a KRAS-associated disease or disorder in a subject comprising administering to the subject a therapeutically-effective amount of a KRAS nucleic acid inhibitor molecule and a therapeutically-effective amount of an MEK inhibitor. In certain embodiments, the MEK inhibitor is trametinib. In certain embodiments, KRAS-associated disease or disorder is a KRAS-associated cancer. In certain embodiments, the KRAS-associated cancer is resistant to treatment with the MEK inhibitor or the immunotherapeutic agent prior to administration of the KRAS nucleic acid inhibitor molecule.

Also disclosed is a method of treating a KRAS-associated disease or disorder, such as cancer, in a subject comprising administering to the subject a therapeutically-effective amount of a KRAS nucleic acid inhibitor molecule and a therapeutically-effective amount of an immunotherapeutic agent. A related aspect is directed to a method of potentiating a therapeutic effect of an immunotherapeutic agent against a KRAS-associated disease or disorder, such as cancer, comprising administering to a subject having the KRAS-associated cancer a KRAS nucleic acid inhibitor molecule in an amount sufficient to potentiate the therapeutic effect of the immunotherapeutic agent against the cancer.

In certain embodiments, prior to administering the KRAS nucleic acid inhibitor molecule, the KRAS-associated cancer is associated with a non-T cell inflamed phenotype that is resistant to immunotherapy and administering the KRAS nucleic acid inhibitor molecule converts the non-T cell inflamed phenotype into a T cell-inflamed phenotype that is responsive to an immunotherapeutic agent.

In certain embodiments, the methods disclosed herein further comprise administering an agent that reduces stromal markers in the tumor microenvironment, such as a TGF-β inhibitor or a CSF1 inhibitor.

In certain embodiments, the immunotherapeutic agent is an antagonist of an inhibitory immune checkpoint molecule or an agonist of a co-stimulatory checkpoint molecule. In certain embodiments, the immunotherapeutic agent is an antagonist of an inhibitory check point, and the inhibitory check point is PD-1 or PD-L1, and in certain embodiments, the antagonist of the inhibitory immune checkpoint molecule or the agonist of the co-stimulatory checkpoint molecule is a monoclonal antibody.

In certain embodiments, the KRAS-associated cancer is pancreatic cancer.

According to various embodiments, the KRAS nucleic acid inhibitor molecule is a double stranded RNAi inhibitor molecule comprising a sense stand and an antisense strand and a region of complementarity between the sense strand and the antisense strand of about 15-45 base pairs. In certain embodiments, the sense strand is 25-40 nucleotides and contains a stem and a loop, and the antisense strand is 18-24 nucleotides and optionally comprises a single-stranded overhang of 1-2 nucleotides at its 3′-terminus, wherein the sense strand and antisense strand form a duplex region of 18-24 base pairs.

In certain embodiments, the sense strand comprises or consists of the sequence of SEQ ID NO: 13 and/or the antisense strand comprises or consists of the sequence of SEQ ID NO: 14 or 18. In certain embodiments, the sense strand comprises or consists of the sequence of one of SEQ ID NO: 15 and/or the antisense strand comprises or consists of the sequence of one of SEQ ID NO: 16 or 19. In certain embodiments, the sense strand comprises or consists of the sequence of SEQ ID NO: 13, and the antisense strand comprises or consists of the sequence of SEQ ID NO: 14. In certain embodiments, the sense strand comprises or consists of the sequence of SEQ ID NO: 13, and the antisense strand comprises or consists of the sequence of SEQ ID NO: 18. In certain embodiments, the sense strand comprises or consists of the sequence of SEQ ID NO: 15, and the antisense strand comprises or consists of the sequence of SEQ ID NO: 16. In certain embodiments, the sense strand comprises or consists of the sequence of SEQ ID NO: 15, and the antisense strand comprises or consists of the sequence of SEQ ID NO: 19. In certain embodiments, the sense strand comprises or consists of the sequence of SEQ ID NO: 7, and the antisense strand comprises or consists of the sequence of SEQ ID NO: 17. Other nucleic acid inhibitor molecules are also contemplated, as disclosed elsewhere in the application.

### BRIEF DESCRIPTION OF THE DRAWINGS

The accompanying drawings, which are incorporated in and constitute a part of this specification, illustrate certain embodiments, and together with the written description, serve to explain certain principles of the compositions and methods disclosed herein.

FIG. 1 shows the structure and nucleotide sequences for 12 different KRAS DsiRNA constructs (SEQ ID NOS 1-2, 20-25, 5-6, 26-27, 3-4 and 28-37, respectively, in order of appearance) and their corresponding tetraloop structure and nucleotide sequences (SEQ ID NOS 7-8, 38-43, 11-12, 44-45, 9-10 and 46-55, respectively, in order of appearance), as well as a corresponding U/GG tetraloop structure and nucleotide sequence for KRAS-446 (SEQ ID NOS 15 and 19, respectively, in order of appearance). The tetraloop structures include a sense strand of 36 nucleotides and a separate antisense strand of 22 nucleotides. The arrow in the tetraloop structures indicates the location of the discontinuity between the sense and antisense strands, where the “C” on the right hand side of the arrow is the 3′-end of the sense strand and the “U,” “A,” or “G” nucleotide on the left hand side of the arrow is the 5′-end of the antisense strand.

FIG. 2A is a graph showing KRAS mRNA expression levels 24 hours after a single treatment cycle with various constructs of KRAS DsiRNA, as shown in FIG. 1, at 1 nM in MIA PaCa cells, as described in Example 1.

FIG. 2B is a graph showing KRAS mRNA expression levels 24 hours after a single treatment cycle with various constructs of KRAS DsiRNA, as shown in FIG. 1, at 0.1 nM in MIA PaCa cells, as described in Example 1.

FIG. 3A shows the structure and nucleotide sequences for 3 different KRAS tetraloop constructs: KRAS-194T (SEQ ID NOS 7 and 17, respectively, in order of appearance), KRAS-465T (SEQ ID NOS 13 and 18, respectively, in order of appearance), and KRAS-446T (SEQ ID NOS 15 and 19, respectively, in order of appearance), as well as the structure and nucleotide sequence for KRAS-465T/MOP (SEQ ID NOS 13-14, respectively, in order of appearance), a tetraloop construct containing a 4′-oxymethylphosphonate modification at nucleotide 1 of the antisense strand (also referred to as “KRAS1”). The tetraloop structures include a sense strand of 36 nucleotides and a separate antisense strand of 22 nucleotides. The arrow in the tetraloop structures indicates the location of the discontinuity between the sense and antisense strands, where the “C” on the right hand side of the arrow is the 3′-end of the sense strand and the “U” on the left hand side of the arrow is the 5′-end of the antisense strand.

FIG. 3B is a column scatter plot showing KRAS mRNA expression levels in a mouse tumor model using MIA PaCa2 tumor cells 24 hours after three-day daily administration of 3 mg/kg of KRAS nucleic acid inhibitor molecules as described in Example 1 and shown in FIG. 3A.

FIG. 3C is a column scatter plot showing KRAS mRNA expression levels in a mouse tumor model using MIA LS411N tumor cells 24 hours after three-day daily administration of 3 mg/kg of KRAS nucleic acid inhibitor molecules as described in Example 1 and shown in FIG. 3A.

FIG. 4A shows the structure and nucleotide sequences for the KRAS tetraloop constructs KRAS-465T/MOP (SEQ ID NOS 13-14, respectively, in order of appearance) and KRAS-446T/MOP (SEQ ID NOS 15-16, respectively, in order of appearance), which contain a 4′-oxymethylphosphonate modification at nucleotide 1 of the antisense strand. The tetraloop structures include a sense strand of 36 nucleotides and a separate antisense strand of 22 nucleotides. The arrow in the tetraloop structures indicates the location of the discontinuity between the sense and antisense strands, where the “C” on the right hand side of the arrow is the 3′-end of the sense strand and the “U” on the left hand side of the arrow is the 5′-end of the antisense strand.

FIG. 4B is a column scatter plot showing KRAS mRNA expression levels in a mouse tumor model LS411N tumors at 24 hour and 72 hour time points after three-day daily administration of 3 mg/kg of two KRAS nucleic acid inhibitor molecules (KRAS-465T/MOP and KRAS-446T/MOP) as described in Example 1 and shown in FIG. 4A.

FIG. 5A shows the treatment schedule for C57BL/6 mice implanted with murine PDAC Pan02 tumors and treated with a KRAS nucleic acid inhibitor molecule formulated in an LNP (“KRAS/LNP”), as described in Example 3.

FIG. 5B are column scatter plots showing Kras, Cd8, FoxP3, and CXCL1 mRNA expression levels for C57BL/6 mice implanted with murine PDAC Pan02 tumors and treated with KRAS/LNP, as described in Example 3.

FIG. 6 is a graph showing tumor volume over time for Pan02 tumors implanted in C57BL/6 mice and treated with KRAS/LNP, as described in Example 3.

FIG. 7 is a graph showing tumor volume over time for Panc1 tumors implanted in C57BL/6 mice and treated with KRAS/LNP, as described in Example 3.

FIG. 8A is a column scatter plot showing CXCL1 mRNA expression levels for C57BL/6 mice implanted with murine PDAC Pan02 tumors and treated with KRAS/LNP, as described in Example 4.

FIG. 8B is a column scatter plot showing FoxP3 mRNA expression levels for C57BL/6 mice implanted with murine PDAC Pan02 tumors and treated with KRAS/LNP, as described in Example 4.

FIG. 8C is a column scatter plot showing Cd8 mRNA expression levels for C57BL/6 mice implanted with murine PDAC Pan02 tumors and treated with KRAS/LNP, as described in Example 4.

FIG. 8D is a column scatter plot showing ROBO1 mRNA expression levels for C57BL/6 mice implanted with murine PDAC Pan02 tumors and treated with KRAS/LNP, as described in Example 4.

FIG. 8E is a column scatter plot showing TGF-β mRNA expression levels for C57BL/6 mice implanted with murine PDAC Pan02 tumors and treated with KRAS/LNP, as described in Example 4.

FIG. 8F is a column scatter plot showing CXCL5 mRNA expression levels for C57BL/6 mice implanted with murine PDAC Pan02 tumors and treated with KRAS/LNP, as described in Example 4.

FIG. 8G is a column scatter plot showing IL-10 mRNA expression levels for C57BL/6 mice implanted with murine PDAC Pan02 tumors and treated with KRAS/LNP, as described in Example 4.

FIG. 8H is a column scatter plot showing Cd274 (PD-L1) mRNA expression levels for C57BL/6 mice implanted with murine PDAC Pan02 tumors and treated with KRAS/LNP, as described in Example 4.

FIG. 8I is a column scatter plot showing Axin2 mRNA expression levels for C57BL/6 mice implanted with murine PDAC Pan02 tumors and treated with KRAS/LNP, as described in Example 4.

FIG. 8J is a column scatter plot showing CSF3 mRNA expression levels for C57BL/6 mice implanted with murine PDAC Pan02 tumors and treated with KRAS/LNP, as described in Example 4.

FIG. 9A is a column scatter plot showing Cd8 mRNA expression levels for C57BL/6 mice implanted with murine PDAC Pan02 tumors and treated with trametinib and KRAS/LNP, as described in Example 5.

FIG. 9B is a column scatter plot showing FoxP3 mRNA expression levels for C57BL/6 mice implanted with murine PDAC Pan02 tumors and treated with trametinib and KRAS/LNP, as described in Example 5.

FIG. 9C is a column scatter plot showing PD-L1 mRNA expression levels for C57BL/6 mice implanted with murine PDAC Pan02 tumors and treated with trametinib and KRAS/LNP, as described in Example 5.

FIG. 10A is a graph showing tumor volume over time for Pan02 tumors implanted in C57BL/6 mice and treated with trametinib (MEKi) and KRAS/LNP, as described in Example 5.

FIG. 10B is a column scatter plot showing FoxP3 expression levels for C57BL/6 mice implanted with PDAC Pan02 tumors and treated with tratmetinib (MEKi) alone and with both trametinib (MEKi) and KRAS/LNP, as described in Example 5.

FIG. 10C is a column scatter plot showing CXCL5 mRNA expression levels for C57BL/6 mice implanted with PDAC Pan02 tumors and treated with tratmetinib (MEKi) alone and with both trametinib (MEKi) and KRAS/LNP, as described in Example 5.

FIG. 10D is a column scatter plot showing Cd274 (PD-L1) mRNA expression levels for C57BL/6 mice implanted with PDAC Pan02 tumors and treated with tratmetinib (MEKi) alone and with both trametinib (MEKi) and KRAS/LNP, as described in Example 5.

FIG. 10E is a column scatter plot showing CXCL1 mRNA expression levels for C57BL/6 mice implanted with PDAC Pan02 tumors and treated with tratmetinib (MEKi) alone and with both trametinib (MEKi) and KRAS/LNP, as described in Example 5.

FIG. 10F is a column scatter plot showing Cd8 mRNA expression levels for C57BL/6 mice implanted with PDAC Pan02 tumors and treated with tratmetinib (MEKi) alone and with both trametinib (MEKi) and KRAS/LNP, as described in Example 5.

FIG. 11 shows by immunohistochemistry that combining treatment with KRAS DsiRNA and an MEK inhibitor leads to reduced FoxP3 expression and increased CD8 expression in Pan02 tumors, as discussed in Example 5.

FIG. 12A is a graph showing tumor volume over time for Panc1 tumors implanted into C57BL/6 mice and treated with trametinib and KRAS1, as described in Example 6.

FIG. 12B is a column scatter plot showing Kras and Cd274 (PD-L1) mRNA expression levels for tratmetinib-resistant human PDAC Panc1 tumors treated with tratmetinib alone and treated with both trametinib and KRAS/LNP, as described in Example 6.

FIG. 13 is a graph showing tumor volume overtime for Panc1 tumors implanted into C57BL/6 mice and treated with gemcitabine and KRAS/LNP, as described in Example 6.

FIG. 14 is a graph showing tumor volume over time for Pan02 tumors implanted into C57BL/6 mice and treated with gemcitabine and KRAS/LNP, as described in Example 6.

FIG. 15A is a column scatter plot showing FoxP3 mRNA expression levels for gemcitabine-resistant murine PDAC Pan02 tumors treated with gemcitabine alone and treated with both gemcitabine and KRAS/LNP, as described in Example 6.

FIG. 15B is a column scatter plot showing CXCL1 mRNA expression levels for gemcitabine-resistant murine PDAC Pan02 tumors treated with gemcitabine alone and treated with both gemcitabine and KRAS/LNP, as described in Example 6.

FIG. 15C is a column scatter plot showing Cd8 mRNA expression levels for gemcitabine-resistant murine PDAC Pan02 tumors treated with gemcitabine alone and treated with both gemcitabine and KRAS/LNP, as described in Example 6.

FIG. 15D is a column scatter plot showing Cd274 (PD-L1) mRNA expression levels for gemcitabine-resistant murine PDAC Pan02 tumors treated with gemcitabine alone and treated with both gemcitabine and KRAS/LNP, as described in Example 6.

FIG. 15E is a column scatter plot showing ROBO1 mRNA expression levels for gemcitabine-resistant murine PDAC Pan02 tumors treated with gemcitabine alone and treated with both gemcitabine and KRAS/LNP, as described in Example 6.

FIG. 15F is a column scatter plot showing TGF-3 mRNA expression levels for gemcitabine-resistant murine PDAC Pan02 tumors treated with gemcitabine alone and treated with both gemcitabine and KRAS/LNP, as described in Example 6.

FIG. 15G is a column scatter plot showing Axin2 mRNA expression levels for gemcitabine-resistant murine PDAC Pan02 tumors treated with gemcitabine alone and treated with both gemcitabine and KRAS/LNP, as described in Example 6.

FIG. 16A is a graph showing tumor volume over time for Pan02 tumors implanted into C57BL/6 mice and treated with a TGF-β inhibitor, as described in Example 7.

FIG. 16B is a graph showing tumor volume over time for Pan02 tumors implanted into C57BL/6 mice and treated with a CSF1 antibody, as described in Example 7.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Administer: As used herein, “administering” a composition to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Antisense strand: A double stranded nucleic acid inhibitor molecule comprises two oligonucleotide strands: an antisense strand and a sense strand. The antisense strand or a region thereof is partially, substantially or fully complementary to a corresponding region of a target nucleic acid. In addition, the antisense strand of the double stranded nucleic acid inhibitor molecule or a region thereof is partially, substantially or fully complementary to the sense strand of the double stranded nucleic acid inhibitor molecule or a region thereof. In certain embodiments, the antisense strand may also contain nucleotides that are non-complementary to the target nucleic acid sequence. The non-complementary nucleotides may be on either side of the complementary sequence or may be on both sides of the complementary sequence. In certain embodiments, where the antisense strand or a region thereof is partially or substantially complementary to the sense strand or a region thereof, the non-complementary nucleotides may be located between one or more regions of complementarity (e.g., one or more mismatches). The antisense strand of a double stranded nucleic acid inhibitor molecule is also referred to as the guide strand.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Bicyclic nucleotide: As used herein, the term “bicyclic nucleotide” refers to a nucleotide comprising a bicyclic sugar moiety.

Bicyclic sugar moiety: As used herein, the term “bicyclic sugar moiety” refers to a modified sugar moiety comprising a 4 to 7 membered ring (including but not limited to a furanosyl) comprising a bridge connecting two atoms of the 4 to 7 membered ring to form a second ring, resulting in a bicyclic structure. Typically, the 4 to 7 membered ring is a sugar. In some embodiments, the 4 to 7 member ring is a furanosyl. In certain embodiments, the bridge connects the 2′-carbon and the 4′-carbon of the furanosyl.

Complementary: As used herein, the term “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. “Fully complementary” or 100% complementarity refers to the situation in which each nucleotide monomer of a first oligonucleotide strand or of a segment of a first oligonucleotide strand can form a base pair with each nucleotide monomer of a second oligonucleotide strand or of a segment of a second oligonucleotide strand. Less than 100% complementarity refers to the situation in which some, but not all, nucleotide monomers of two oligonucleotide strands (or two segments of two oligonucleotide strands) can form base pairs with each other. “Substantial complementarity” refers to two oligonucleotide strands (or segments of two oligonucleotide strands) exhibiting 90% or greater complementarity to each other. “Sufficiently complementary” refers to complementarity between a target mRNA and a nucleic acid inhibitor molecule, such that there is a reduction in the amount of protein encoded by a target mRNA.

Complementary strand: As used herein, the term “complementary strand” refers to a strand of a double stranded nucleic acid inhibitor molecule that is partially, substantially or fully complementary to the other strand.

Deoxyribofuranosyl: As used herein, the term “deoxyribofuranosyl” refers to a furanosyl that is found in naturally occurring DNA and has a hydrogen group at the 2′-carbon, as illustrated below:

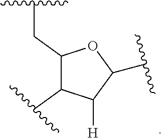 [SMILES: *CC1OC(*)CC1*]

Deoxyribonucleotide: As used herein, the term “deoxyribonucleotide” refers to a natural nucleotide (as defined herein) or a modified nucleotide (as defined herein), which has a hydrogen group at the 2′-position of the sugar moiety.

dsRNAi inhibitor molecule: As used herein, the term “dsRNAi inhibitor molecule” refers to a double-stranded nucleic acid inhibitor molecule having a sense strand (passenger) and antisense strand (guide), where the antisense strand or part of the antisense strand is used by the Argonaute 2 (Ago2) endonuclease in the cleavage of a target mRNA.

Duplex: As used herein, the term “duplex,” in reference to nucleic acids (e.g., oligonucleotides), refers to a structure formed through complementary base pairing of two antiparallel sequences of nucleotides.

Excipient: As used herein, the term “excipient” refers to a non-therapeutic agent that may be included in a composition, for example to provide or contribute to a desired consistency or stabilizing effect.

Furanosyl: As used herein, the term “furanosyl” refers to a structure comprising a 5-membered ring with four carbon atoms and one oxygen atom.

Internucleotide linking group: As used herein, the term “internucleotide linking group” or “internucleotide linkage” refers to a chemical group capable of covalently linking two nucleoside moieties. Typically, the chemical group is a phosphorus-containing linkage group containing a phospho or phosphite group. Phospho linking groups are meant to include a phosphodiester linkage, a phosphorodithioate linkage, a phosphorothioate linkage, a phosphotriester linkage, a thionoalkylphosphonate linkage, a thionalkylphosphotriester linkage, a phosphoramidite linkage, a phosphonate linkage and/or a boranophosphate linkage. Many phosphorus-containing linkages are well known in the art, as disclosed, for example, in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050. In other embodiments, the oligonucleotide contains one or more internucleotide linking groups that do not contain a phosphorous atom, such short chain alkyl or cycloalkyl internucleotide linkages, mixed heteroatom and alkyl or cycloalkyl internucleotide linkages, or one or more short chain heteroatomic or heterocyclic internucleotide linkages, including, but not limited to, those having siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; and amide backbones. Non-phosphorous containing linkages are well known in the art, as disclosed, for example, in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

Immune checkpoint molecules: As used herein, the term “immune checkpoint molecule” refers to molecules on immune cells, such as T cells, that are important under normal physiological conditions for the maintenance of self-tolerance (or the prevention of autoimmunity) and the protection of host cells and tissue when the immune system responds to a foreign pathogen. Certain immune checkpoint molecules are co-stimulatory molecules that amplify a signal involved in the T cell response to antigen while certain immune checkpoint molecules are inhibitory molecules (e.g., CTLA-4 or PD-1) that reduce a signal involved in the T cell response to antigen.

Immunotherapeutic agent: an agent for the treatment of a disease or disorder, such as cancer, that acts to enhance the immune system's ability to fight the disease or disorder. Examples of immunotherapeutic agents include checkpoint inhibitors, antibodies, and cytokines such as interferons and interleukins.

KRAS-associated disease or disorder: As used herein, the term “KRAS-associated disease or disorder” refers to a disease or disorder that is associated with altered KRAS expression, level, and/or activity. Notably, a “KRAS-associated disease or disorder” includes cancer and/or proliferative diseases, conditions, or disorders.

Loop: As used herein, the term “loop” refers to a structure formed by a single strand of a nucleic acid, in which complementary regions that flank a particular single stranded nucleotide region hybridize in a way that the single stranded nucleotide region between the complementary regions is excluded from duplex formation or Watson-Crick base pairing. A loop is a single stranded nucleotide region of any length. Examples of loops include the unpaired nucleotides present in such structures as hairpins and tetraloops.

MEKInhibitor: As used herein, the term “MEK inhibitor” refers to a compound or agent that reduces an activity of the mitogen-activated protein kinase kinase enzyme MEK1 and/or MEK2.

Modified nucleobase: As used herein, the term “modified nucleobase” refers to any nucleobase that is not a natural nucleobase or a universal nucleobase. Suitable modified nucleobases include diaminopurine and its derivatives, alkylated purines or pyrimidines, acylated purines or pyrimidines thiolated purines or pyrimidines, and the like. Other suitable modified nucleobases include analogs of purines and pyrimidines. Suitable analogs include, but are not limited to, 1-methyladenine, 2-methyladenine, N6-methyladenine, N6-isopentyladenine, 2-methylthio-N6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, hypoxanthine, xanthine, 2-aminopurine, 6-hydroxyaminopurine, nitropyrrolyl, nitroindolyl and difluorotolyl, 6-thiopurine and 2,6-diaminopurine nitropyrrolyl, nitroindolyl and difluorotolyl. Typically a nucleobase contains a nitrogenous base. In certain embodiments, the nucleobase does not contain a nitrogen atom. See e.g., U.S. Published Patent Application No. 20080274462.

Modified nucleoside: As used herein, the term “modified nucleoside” refers to a heterocyclic nitrogenous base in N-glycosidic linkage with a sugar (e.g., deoxyribose or ribose or analog thereof) that is not linked to a phosphate group or a modified phosphate group (as defined herein) and that contains one or more of a modified nucleobase (as defined herein), a universal nucleobase (as defined herein) or a modified sugar moiety (as defined herein). The modified or universal nucleobases (also referred to herein as base analogs) are generally located at the 1′-position of a nucleoside sugar moiety and refer to nucleobases other than adenine, guanine, cytosine, thymine and uracil at the 1′-position. In certain embodiments, the modified or universal nucleobase is a nitrogenous base. In certain embodiments, the modified nucleobase does not contain nitrogen atom. See e.g., U.S. Published Patent Application No. 20080274462. In certain embodiments, the modified nucleotide does not contain a nucleobase (abasic). Suitable modified or universal nucleobases or modified sugars in the context of the present disclosure are described herein.

Modified nucleotide: As used herein, the term “modified nucleotide” refers to a heterocyclic nitrogenous base in N-glycosidic linkage with a sugar (e.g., ribose or deoxyribose or analog thereof) that is linked to a phosphate group or a modified phosphate group (as defined herein) and contains one or more of a modified nucleobase (as defined herein), a universal nucleobase (as defined herein), or a modified sugar moiety (as defined herein). The modified or universal nucleobases (also referred to herein as base analogs) are generally located at the 1′-position of a nucleoside sugar moiety and refer to nucleobases other than adenine, guanine, cytosine, thymine and uracil at the 1′-position. In certain embodiments, the modified or universal nucleobase is a nitrogenous base. In certain embodiments, the modified nucleobase does not contain nitrogen atom. See e.g., U.S. Published Patent Application No. 20080274462. In certain embodiments, the modified nucleotide does not contain a nucleobase (abasic). Suitable modified or universal nucleobases, modified sugar moieties, or modified phosphate groups in the context of the present disclosure are described herein.

Modified phosphate group: As used herein, the term “modified phosphate group” refers to a modification of the phosphate group that does not occur in natural nucleotides and includes non-naturally occurring phosphate mimics as described herein, including phosphate mimics that include a phosphorous atom and anionic phosphate mimics that do not include phosphate (e.g. acetate). Modified phosphate groups also include non-naturally occurring internucleotide linking groups, including both phosphorous-containing internucleotide linking groups, including, for example, phosphorothioate, and non-phosphorous containing linking groups, as described herein.

Modified sugar moiety: As used herein, a “modified sugar moiety” refers to a substituted sugar moiety (as defined herein) or a sugar analog (as defined herein).

Natural nucleobase: As used herein, the term “natural nucleobase” refers to the five primary, naturally occurring heterocyclic nucleobases of RNA and DNA, i.e., the purine bases: adenine (A) and guanine (G), and the pyrimidine bases: thymine (T), cytosine (C), and uracil (U).

Natural nucleoside: As used herein, the term “natural nucleoside” refers to a natural nucleobase (as defined herein) in N-glycosidic linkage with a natural sugar moiety (as defined herein) that is not linked to a phosphate group.

Natural nucleotide: As used herein, the term “natural nucleotide” refers to a natural nucleobase (as defined herein) in N-glycosidic linkage with a natural sugar moiety (as defined herein) that is linked to a phosphate group.

Natural sugar moiety: As used herein, the term “natural sugar moiety” refers to a ribofuranosyl (as defined herein) or a deoxyribofuranosyl (as defined herein).

Non-T cell inflamed phenotype: As used herein, “non-T cell inflamed phenotype” refers to a tumor microenvironment without a pre-existing T cell response against the tumor, as evidenced by little to no accumulation of infiltrating CD8+ T cells in the tumor microenvironment. Typically, the non-T cell inflamed phenotype is also characterized by a limited chemokine profile that does not promote the recruitment and accumulation of CD8+ T cells in the tumor microenvironment and/or a minimal or absent type I IFN gene signature.

Nucleic acid inhibitor molecule: As used herein, the term “nucleic acid inhibitor molecule” refers to an oligonucleotide molecule that reduces or eliminates the expression of a target gene wherein the oligonucleotide molecule contains a region that specifically targets a sequence in the target gene mRNA. Typically, the targeting region of the nucleic acid inhibitor molecule comprises a sequence that is sufficiently complementary to a sequence on the target gene mRNA to direct the effect of the nucleic acid inhibitor molecule to the specified target gene. For example, a “KRAS nucleic acid inhibitor molecule” reduces or eliminates the expression of a KRAS gene. The nucleic acid inhibitor molecule may include ribonucleotides, deoxyribonucleotides, and/or modified nucleotides.

Nucleobase: As used herein, the term “nucleobase” refers to a natural nucleobase (as defined herein), a modified nucleobase (as defined herein), or a universal nucleobase (as defined herein).

Nucleoside: As used herein, the term “nucleoside” refers to a natural nucleoside (as defined herein) or a modified nucleoside (as defined herein).

Nucleotide: As used herein, the term “nucleotide” refers to a natural nucleotide (as defined herein) or a modified nucleotide (as defined herein).

Overhang: As used herein, the term “overhang” refers to terminal non-base pairing nucleotide(s) at either end of either strand of a double-stranded nucleic acid inhibitor molecule. In certain embodiments, the overhang results from one strand or region extending beyond the terminus of the complementary strand to which the first strand or region forms a duplex. One or both of two oligonucleotide regions that are capable of forming a duplex through hydrogen bonding of base pairs may have a 5′- and/or 3′-end that extends beyond the 3′- and/or 5′-end of complementarity shared by the two polynucleotides or regions. The single-stranded region extending beyond the 3′- and/or 5′-end of the duplex is referred to as an overhang.

Pharmaceutical composition: As used herein, the term “pharmaceutical composition” comprises a pharmacologically effective amount of a double-stranded nucleic acid inhibitor molecule and a pharmaceutically acceptable excipient (as defined herein).

Pharmaceutically acceptable excipient: As used herein, the term “pharmaceutically acceptable excipient” means that the excipient is one that is suitable for use with humans and/or animals without undue adverse side effects (such as toxicity, irritation, and allergic response) commensurate with a reasonable benefit/risk ratio.

Phosphate mimic: As used herein, the term “phosphate mimic” refers to a chemical moiety at the 5′-terminal end of an oligonucleotide that mimics the electrostatic and steric properties of a phosphate group. Many phosphate mimics have been developed that can be attached to the 5′-end of an oligonucleotide (see, e.g., U.S. Pat. No. 8,927,513; Prakash et al. Nucleic Acids Res., 2015, 43(6).2993-3011). Typically, these 5′-phosphate mimics contain phosphatase-resistant linkages. Suitable phosphate mimics include 5′-phosphonates, such as 5′-methylenephosphonate (5′-MP) and 5′-(E)-vinylphosphonate (5′-VP) and 4′-phosphate analogs that are bound to the 4′-carbon of the sugar moiety (e.g., a ribose or deoxyribose or analog thereof) of the 5′-terminal nucleotide of an oligonucleotide, such as 4′-oxymethylphosphonate, 4′-thiomethylphosphonate, or 4′-aminomethylphosphonate, as described in International Publication No. WO 2018/045317, which is hereby incorporated by reference in its entirety. In certain embodiments, the 4′-oxymethylphosphonate is represented by the formula —O—CH₂—PO(OH)₂ or —O—CH₂—PO(OR)₂, where R is independently selected from H, CH₃, an alkyl group, or a protecting group. In certain embodiments, the alkyl group is CH₂CH₃. More typically, R is independently selected from H, CH₃, or CH₂CH₃. Other modifications have been developed for the 5′-end of oligonucleotides (see, e.g., WO 2011/133871).

Potentiate: The term “potentiate” or “potentiating” as used herein refers to the ability of one therapeutic agent (e.g., a KRAS nucleic acid inhibitor molecule) to increase or enhance the therapeutic effect of another therapeutic agent (e.g., an MEK inhibitor or an immunotherapeutic agent).

Proliferative disease or cancer: The term “proliferative disease” or “cancer” as used herein refers to a disease, condition, trait, genotype or phenotype characterized by unregulated cell growth or replication as is known in the art, including leukemias, for example, acute myelogenous leukemia (AML), chronic myelogenous leukemia (CML), acute lymphocytic leukemia (ALL), and chronic lymphocytic leukemia; AIDS-related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, colorectal cancer, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and other cancer or proliferative disease, condition, trait, genotype or phenotype that can respond to the modulation of disease related gene expression in a cell or tissue, alone or in combination with other therapies.

Protecting group: As used herein, the term “protecting group” is used in the conventional chemical sense as a group which reversibly renders unreactive a functional group under certain conditions of a desired reaction. After the desired reaction, protecting groups may be removed to deprotect the protected functional group. All protecting groups should be removable under conditions which do not degrade a substantial proportion of the molecules being synthesized.

Reduce(s): The term “reduce” or “reduces” as used herein refers to its meaning as is generally accepted in the art. With reference to nucleic acid inhibitor molecules, the term generally refers to the reduction in the expression of a gene, or level of RNA molecules or equivalent RNA molecules encoding one or more proteins or protein subunits, or activity of one or more proteins or protein subunits, below that observed in the absence of the nucleic acid inhibitor molecules or inhibitor.

Resistance: The term “resistance” as used herein refers to the condition that occurs when a treatment that previously reduced or inhibited tumor growth in a subject no longer reduces or inhibits tumor growth in that subject.

Ribofuranosyl: As used herein, the term “ribofuranosyl” refers to a furanosyl that is found in naturally occurring RNA and has a hydroxyl group at the 2′-carbon, as illustrated below:

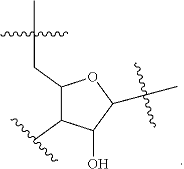 [SMILES: *CC1OC(*)C(O)C1*]

Ribonucleotide: As used herein, the term “ribonucleotide” refers to a natural nucleotide (as defined herein) or a modified nucleotide (as defined herein) which has a hydroxyl group at the 2′-position of the sugar moiety.

Sense strand: A double-stranded nucleic acid inhibitor molecule comprises two oligonucleotide strands: an antisense strand and a sense strand. The sense strand or a region thereof is partially, substantially or fully complementary to the antisense strand of the double-stranded nucleic acid inhibitor molecule or a region thereof. In certain embodiments, the sense strand may also contain nucleotides that are non-complementary to the antisense strand. The non-complementary nucleotides may be on either side of the complementary sequence or may be on both sides of the complementary sequence. In certain embodiments, where the sense strand or a region thereof is partially or substantially complementary to the antisense strand or a region thereof, the non-complementary nucleotides may be located between one or more regions of complementarity (e.g., one or more mismatches). The sense strand is also called the passenger strand.

Subject: As used herein, the term “subject” means any mammal, including mice, rabbits, and humans. In one embodiment, the subject is a human. The terms “individual” or “patient” are intended to be interchangeable with “subject.”

Substituted sugar moiety: As used herein, a “substituted sugar moiety” includes furanosyls comprising one or more modifications. Typically, the modifications occur at the 2′-, 3′-, 4′-, or 5′-carbon position of the sugar. In certain embodiments, the substituted sugar moiety is a bicyclic sugar moiety comprising a bridge that connects the 2′-carbon with the 4-carbon of the furanosyl.

Sugar analog: As used herein, the term “sugar analog” refers to a structure that does not comprise a furanosyl and that is capable of replacing the naturally occurring sugar moiety of a nucleotide, such that the resulting nucleotide is capable of (1) incorporation into an oligonucleotide and (2) hybridization to a complementary nucleotide. Such structures typically include relatively simple changes to the furanosyl, such as rings comprising a different number of atoms (e.g., 4, 6, or 7-membered rings); replacement of the oxygen of the furanosyl with a non-oxygen atom (e.g., carbon, sulfur, or nitrogen); or both a change in the number of atoms and a replacement of the oxygen. Such structures may also comprise substitutions corresponding with those described for substituted sugar moieties. Sugar analogs also include more complex sugar replacements (e.g., the non-ring systems of peptide nucleic acid). Sugar analogs include without limitation morpholinos, cyclohexenyls and cyclohexitols.

Sugar moiety: As used herein, the term “sugar moiety” refers to a natural sugar moiety or a modified sugar moiety of a nucleotide or nucleoside.

T cell-inflamed tumor phenotype: As used herein, “T cell-inflamed phenotype” refers to a tumor microenvironment with a pre-existing T cell response against the tumor, as evidenced by an accumulation of infiltrating CD8+ T cells in the tumor microenvironment. Typically, the T cell-inflamed phenotype is also characterized by a broad chemokine profile capable of recruiting CD8+ T cells to the tumor microenvironment (including CXCL9 and/or CXCL10) and/or a type I IFN gene signature.

Tetraloop: As used herein, the term “tetraloop” refers to a loop (a single stranded region) that forms a stable secondary structure that contributes to the stability of an adjacent Watson-Crick hybridized nucleotides. Without being limited to theory, a tetraloop may stabilize an adjacent Watson-Crick base pair by stacking interactions. In addition, interactions among the nucleotides in a tetraloop include but are not limited to non-Watson-Crick base pairing, stacking interactions, hydrogen bonding, and contact interactions (Cheong et al., Nature, 1990, 346(6285):680-2; Heus and Pardi, Science, 1991, 253(5016):191-4). A tetraloop confers an increase in the melting temperature (Tm) of an adjacent duplex that is higher than expected from a simple model loop sequence consisting of random bases. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. A tetraloop may contain ribonucleotides, deoxyribonucleotides, modified nucleotides, and combinations thereof. In certain embodiments, a tetraloop consists of four nucleotides. In certain embodiments, a tetraloop consists of five nucleotides.

Examples of RNA tetraloops include the UNCG family of tetraloops (e.g., UUCG), the GNRA family of tetraloops (e.g., GAAA), and the CUYG family of tetraloops, including the CUUG tetraloop. (Woese et al., PNAS, 1990, 87(21):8467-71; Antao et al., Nucleic Acids Res., 1991, 19(21):5901-5). Other examples of RNA tetraloops include the GANC, A/UGNN, and UUUM tetraloop families (Thapar et al., Wiley Interdiscip Rev RNA, 2014, 5(1):1-28) and the GGUG, RNYA, and AGNN tetraloop families (Bottaro et al., Biophys J., 2017, 113:257-67). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). (Nakano et al. Biochemistry, 2002, 41(48):14281-14292. Shinji et al., Nippon Kagakkai Koen Yokoshu, 2000, 78(2):731).

Triloop: As used herein, the term “triloop” refers to a loop (a single stranded region) that forms a stable secondary structure that contributes to the stability of an adjacent Watson-Crick hybridized nucleotides and consists of three nucleotides. Without being limited to theory, a triloop may be stabilized by non-Watson-Crick base pairing of nucleotides within the triloop and base-stacking interactions. (Yoshizawa et al., Biochemistry 1997; 36, 4761-4767). A triloop can also confer an increase in the melting temperature (Tm) of an adjacent duplex that is higher than expected from a simple model loop sequence consisting of random bases. A triloop may contain ribonucleotides, deoxyribonucleotides, modified nucleotides, and combinations thereof. Examples of triloops include the GNA family of triloops (e.g., GAA, GTA, GCA, and GGA). (Yoshizawa 1997).

Therapeutically effective amount: As used herein, a “therapeutically effective amount” or “pharmacologically effective amount” refers to that amount of an agent, such as a double-stranded nucleic acid inhibitor molecule, an MEK inhibitor, or an immunotherapeutic agent, effective to produce the intended pharmacological, therapeutic or preventive result.

Universal nucleobase: As used herein, a “universal nucleobase” refers to a base that can pair with more than one of the bases typically found in naturally occurring nucleic acids and can thus substitute for such naturally occurring bases in a duplex. The base need not be capable of pairing with each of the naturally occurring bases. For example, certain bases pair only or selectively with purines, or only or selectively with pyrimidines. The universal nucleobase may base pair by forming hydrogen bonds via Watson-Crick or non-Watson-Crick interactions (e.g., Hoogsteen interactions). Representative universal nucleobases include inosine and its derivatives.

### DETAILED DESCRIPTION

This application provides KRAS nucleic acid inhibitor molecules that can modulate (e.g., inhibit) KRAS expression and methods of treating a KRAS-associated disease or disorder in a subject comprising administering to the subject a therapeutically-effective amount of a KRAS nucleic acid inhibitor molecule. This application further provides methods of treating a KRAS-associated disease or disorder in a subject comprising administering to the subject a therapeutically-effective amount of a KRAS nucleic acid inhibitor and a therapeutically-effective amount of an additional agent, such as an MEK inhibitor or an immunotherapeutic agent. The KRAS nucleic acid inhibitor molecules of the invention modulate KRAS RNAs such as those corresponding to the cDNA sequences referred to by GenBank Accession Nos. NM_033360 and NM_004985, as well as those referred to in U.S. Published Pat. Nos. 8,372,816; 8,513,207; 9,200,284; and 9,809,819 and U.S. Published Patent Application No. 2018/0044680, all of which are incorporated by reference herein.

Also disclosed herein are new methods and compositions for treating cancer, including cancer that is not responsive to immunotherapy (e.g., blockade of immune checkpoint molecules). Typically, cancer that is not responsive to immunotherapy is characterized by a non-T cell inflamed phenotype (also known as cold or non-inflamed tumors), with little to no infiltrating CD8+ T cells in the tumor microenvironment. Reducing KRAS expression can convert a cold or non-inflamed tumor into a hot or inflamed tumor and potentiate the effect of immunotherapy. In other words, by combining a KRAS inhibitor with immunotherapy, it is possible to treat cold or non-inflamed tumors that normally do not respond to immunotherapy. Typically a KRAS nucleic acid inhibitor molecule is used to reduce KRAS expression. However, any KRAS inhibitor or pathway inhibitor that reduces KRAS expression can be used in the methods and compositions described herein, including, but not limited to small molecules, peptides, and antibodies that target KRAS or a component of the KRAS pathway. This combination therapy approach has been shown to potently inhibit tumor growth in vivo.

The below description of the various aspects and embodiments of the invention is provided with reference to exemplary KRAS RNAs, generally referred to herein as KRAS. However, such reference is meant to be exemplary only and the various aspects and embodiments of the invention are also directed to alternate KRAS RNAs, such as mutant KRAS RNAs or additional KRAS splice variants. Certain aspects and embodiments are also directed to other genes involved in KRAS pathways, including genes whose misregulation acts in association with that of KRAS (or is affected or affects KRAS regulation) to produce phenotypic effects that may be targeted for treatment (e.g., tumor formation and/or growth, etc.). Such additional genes can be targeted using DsiRNA and the methods described herein for use of KRAS targeting DsiRNAs. Thus, the inhibition and the effects of such inhibition of the other genes can be performed as described herein.

### Nucleic Acid Inhibitor Molecules

In certain embodiments, KRAS expression is reduced using a nucleic acid inhibitor molecule. Various oligonucleotide structures have been used as nucleic acid inhibitor molecules, including single stranded and double stranded oligonucleotides.

In certain embodiments, the nucleic acid inhibitor molecule is a double-stranded RNAi inhibitor molecule comprising a sense (or passenger) strand and an antisense (or guide) strand. A variety of double stranded RNAi inhibitor molecule structures are known in the art. For example, early work on RNAi inhibitor molecules focused on double-stranded nucleic acid molecules with each strand having sizes of 19-25 nucleotides with at least one 3′-overhang of 1 to 5 nucleotides (see, e.g., U.S. Pat. No. 8,372,968). Subsequently, longer double-stranded RNAi inhibitor molecules that get processed in vivo by the Dicer enzyme to active RNAi inhibitor molecules were developed (see, e.g., U.S. Pat. No. 8,883,996). Later work developed extended double-stranded nucleic acid inhibitor molecules where at least one end of at least one strand is extended beyond the double-stranded targeting region of the molecule, including structures where one of the strands includes a thermodynamically-stabilizing tetraloop structure (see, e.g., U.S. Pat. Nos. 8,513,207, 8,927,705, WO 2010/033225, and WO 2016/100401, which are incorporated by reference for their disclosure of these double-stranded nucleic acid inhibitor molecules). Those structures include single-stranded extensions (on one or both sides of the molecule) and double-stranded extensions.

In some embodiments, the sense and antisense strands range from 15-66, 25-40, or 19-25 nucleotides. In some embodiments, the sense strand is less than 30 nucleotides, such as 19-24 nucleotides, such as 21 nucleotides. In some embodiments, the antisense strand is less than 30 nucleotides, such as 19-24 nucleotides, such as 21, 22, or 23 nucleotides. Typically, the duplex structure is between 15 and 50, such as between 15 and 30, such as between 18 and 26, more typically between 19 and 23, and in certain instances between 19 and 21 base pairs in length.

In some embodiments, the dsRNAi inhibitor molecule may further comprise one or more single-stranded nucleotide overhang(s). Typically, the dsRNAi inhibitor molecule has a single-stranded overhang of 1-4 or 1-2 nucleotides. The single stranded overhang is typically located at the 3′-end of the sense strand and/or the 3′-end of the antisense strand. In certain embodiments, a single-stranded overhang of 1-10, 1-4, or 1-2 nucleotides is located at the 5′-end of the antisense strand. In certain embodiments, a single-stranded overhang of 1-10, 1-4, or 1-2 nucleotides is located at the 5′-end of the sense strand. In certain embodiments, the single-stranded overhang of 1-2 nucleotides is located at the 3′-end of the antisense strand. In certain embodiments, the dsRNA inhibitor molecule has a blunt end, typically at the right hand side of the molecule, i.e., 3′-end of the sense strand and the 5′-end of the antisense strand. In some embodiments, the dsRNA inhibitor molecule has a 2 nucleotide overhang located at the 3′ end of the antisense strand.

In certain embodiments, the dsRNAi inhibitor molecule has a guide strand of 21 nucleotides in length and a passenger strand of 21 nucleotides in length, where there is a two nucleotide 3′-passenger strand overhang on the right side of the molecule (3′-end of passenger strand/5′-end of guide strand) and a two nucleotide 3′-guide strand overhang on the left side of the molecule (5′-end of the passenger strand/3′-end of the guide strand). In such molecules, there is a 19 base pair duplex region.

In certain embodiments, the dsRNAi inhibitor molecule has a guide strand of 23 nucleotides in length and a passenger strand of 21 nucleotides in length, where there is a blunt end on the right side of the molecule (3′-end of passenger strand/5′-end of guide strand) and a two nucleotide 3′-guide strand overhang on the left side of the molecule (5′-end of the passenger strand/3′-end of the guide strand). In such molecules, there is a 21 base pair duplex region.

In certain embodiments, the dsRNAi inhibitor molecule has a guide strand of 23 nucleotides in length and a passenger strand of 21 nucleotides in length, where there is a blunt end on the right side of the molecule (3′-end of passenger strand/5′-end of guide strand) and a two nucleotide 3′-guide strand overhang on the left side of the molecule (5′-end of the passenger strand/3′-end of the guide strand). In such molecules, there is a 21 base pair duplex region.

In certain embodiments, the dsRNAi inhibitor molecule has a guide strand of 27 nucleotides in length and a passenger strand of 25 nucleotides in length, where there is a blunt end on the right side of the molecule (3′-end of passenger strand/5′-end of guide strand) and a two nucleotide 3′-guide strand overhang on the left side of the molecule (5′-end of the passenger strand/3′-end of the guide strand). In such molecules, there is a 25 base pair duplex region.

In some embodiments, the dsRNAi inhibitor molecules include a stem and loop. Typically, a 3′-terminal region or 5′-terminal region of a passenger strand of a dsRNAi inhibitor molecule form a single stranded stem and loop structure.

In some embodiments, the dsRNAi inhibitor molecule contains a stem and a tetraloop or a triloop. In certain embodiments, the dsRNAi inhibitor molecule comprises a guide strand and a passenger strand, wherein the passenger strand contains a stem and tetraloop or triloop and ranges from 20-66 nucleotides in length. Typically, the guide and passenger strands are separate strands, each having a 5′- and 3′-end, that do not form a contiguous oligonucleotide (sometimes referred to as a “nicked” structure).

In certain of those embodiments, the guide strand is between 15 and 40 nucleotides in length. In certain embodiments, the extended part of the passenger strand that contains the stem and tetraloop or triloop is on 3′-end of the strand. In certain other embodiments, the extended part of the passenger strand that contains the stem and tetraloop or triloop is on 5′-end of the strand.

In certain embodiments, the passenger strand of a dsRNAi inhibitor molecule containing a stem and tetraloop is between 26-40 nucleotides in length and the guide strand of the dsRNAi inhibitor molecule contains between 20-24 nucleotides, wherein the passenger strand and guide strand form a duplex region of 18-24 nucleotides. In certain embodiments, the passenger strand is 26-30 nucleotides in length and the stem is 1, 2, or 3 base pairs in length and contains one or more bicyclic nucleotides.

In certain embodiments, the passenger strand of a dsRNAi inhibitor molecule containing a stem and triloop is between 27-39 nucleotides in length and the guide strand of the dsRNAi inhibitor molecule contains between 20-24 nucleotides, wherein the passenger strand and guide strand form a duplex region of 18-24 nucleotides. In certain embodiments, the passenger strand is 27-29 nucleotides in length and the stem is 2 or 3 base pairs in length and contains one or more bicyclic nucleotides.

In certain embodiments, the dsRNAi inhibitor molecule comprises (a) a passenger strand that contains a stem and tetraloop and is 36 nucleotides in length, wherein the first 20 nucleotides of the passenger strand from the 5′-end are complementary to the guide strand and the following 16 nucleotides of the passenger strand form the stem and tetraloop and (b) a guide strand that is 22 nucleotides in length and has a single-stranded overhang of two nucleotides at its 3′-end, wherein the guide and passenger strands are separate strands that do not form a contiguous oligonucleotide.

In certain embodiments, the dsRNAi inhibitor molecule comprises (a) a passenger strand that contains a stem and triloop and is 35 nucleotides in length, wherein the first 20 nucleotides of the passenger strand from the 5′-end are complementary to the guide strand and the following 16 nucleotides of the passenger strand form the stem and triloop and (b) a guide strand that is 22 nucleotides in length and has a single-stranded overhang of two nucleotides at its 3′-end, wherein the guide and passenger strands are separate strands that do not form a contiguous oligonucleotide.

In certain embodiments, the nucleic acid inhibitor molecule is a single-stranded nucleic acid inhibitor molecule. Single stranded nucleic acid inhibitor molecules are known in the art. For example, recent efforts have demonstrated activity of ssRNAi inhibitor molecules (see, e.g., Matsui et al., Molecular Therapy, 2016, 24(5):946-55). And, antisense molecules have been used for decades to reduce expression of specific target genes. Pelechano and Steinmetz, Nature Review Genetics, 2013, 14:880-93. A number of variations on the common themes of these structures have been developed for a range of targets. Single stranded nucleic acid inhibitor molecules include, for example, conventional antisense oligonucleotides, microRNA, ribozymes, aptamers, and ssRNAi inhibitor molecules, all of which are known in the art.

In certain embodiments, the nucleic acid inhibitor molecule is a ssRNAi inhibitor molecule having 14-50, 16-30, or 15-25 nucleotides. In other embodiments, the ssRNAi inhibitor molecule has 18-22 or 20-22 nucleotides. In certain embodiments, the ssRNAi inhibitor molecule has 20 nucleotides. In other embodiments, the ssRNAi inhibitor molecule has 22 nucleotides. In certain embodiments, the nucleic acid inhibitor molecule is a single-stranded oligonucleotide that inhibits exogenous RNAi inhibitor molecules or natural miRNAs.

In certain embodiments, the nucleic acid inhibitor molecule is a single-stranded antisense oligonucleotide having 8-80, 12-50, 12-30, or 12-22 nucleotides. In certain embodiments, the single-stranded antisense oligonucleotide has 16-20, 16-18, 18-22 or 18-20 nucleotides.

### Modifications

Typically, many of the nucleotide subunits of the nucleic acid inhibitor molecules are modified to improve various characteristics of the molecule, such as resistance to nucleases or lowered immunogenicity, (see, e.g., Bramsen et al. (2009), Nucleic Acids Res., 37, 2867-2881). In certain embodiments, from one to every nucleotide of a nucleic acid inhibitor molecule is modified. In certain embodiments, substantially all of the nucleotides of a nucleic acid inhibitor molecule are modified. In certain embodiments, more than half of the nucleotides of a nucleic acid inhibitor molecule are modified. In certain embodiments, less than half of the nucleotides of a nucleic acid inhibitor molecule are modified. In certain embodiments, none of the nucleotides of a nucleic acid inhibitor molecule are modified. Modifications can occur in groups on the oligonucleotide chain or different modified nucleotides can be interspersed.

Many nucleotide modifications have been used in the oligonucleotide field. Modifications can be made on any part of the nucleotide, including the sugar moiety, the phosphoester linkage, and the nucleobase. In certain embodiments of the nucleic acid inhibitor molecule, from one to every nucleotide is modified at the 2′-carbon of the sugar moiety, using, for example, 2′-carbon modifications known in the art and described herein. Typical examples of 2′-carbon modifications include, but are not limited to, 2′-F, 2′-O-methyl (“2′-OMe” or “2′-OCH₃”), 2′-O-methoxyethyl (“2′-MOE” or “2′-OCH₂CH₂OCH₃”). Modifications can also occur at other parts of the sugar moiety of the nucleotide, such as the 5′-carbon, as described herein.

In certain embodiments, the ring structure of the sugar moiety is modified, including, but not limited to, bicyclic nucleotides, such as Locked Nucleic Acids (“LNA”) (see, e.g., Koshkin et al. (1998), Tetrahedron, 54,3607-3630)) and bridged nucleic acids (“BNA”) (see, e.g., U.S. Pat. No. 7,427,672 and Mitsuoka et al. (2009), Nucleic Acids Res., 37(4).1225-38); and Unlocked Nucleic Acids (“UNA”) (see, e.g., Snead et al. (2013), Molecular Therapy-Nucleic Acids, 2,e103(doi: 10.1038/mtna.2013.36)).

Modified nucleobases include nucleobases other than adenine, guanine, cytosine, thymine and uracil at the 1′-position, as known in the art and as described herein. A typical example of a modified nucleobase is 5′-methylcytosine.

The natural occurring internucleotide linkage of RNA and DNA is a 3′ to 5′ phosphodiester linkage. Modified phosphoester linkages include non-naturally occurring internucleotide linking groups, including internucleotide linkages that contain a phosphorous atom and internucleotide linkages that do not contain a phosphorous atom, as known in the art and as described herein. Typically, the nucleic acid inhibitor molecule contains one or more phosphorous-containing internucleotide linking groups, as described herein. In other embodiments, one or more of the internucleotide linking groups of the nucleic acid inhibitor molecule is a non-phosphorus containing linkage, as described herein. In certain embodiments, the nucleic acid inhibitor molecule contains one or more phosphorous-containing internucleotide linking groups and one or more non-phosphorous containing internucleotide linking groups.

The 5′-end of the nucleic acid inhibitor molecule can include a natural substituent, such as a hydroxyl or a phosphate group. In certain embodiments, a hydroxyl group is attached to the 5′-terminal end of the nucleic acid inhibitor molecule. In certain embodiments, a phosphate group is attached to the 5′-terminal end of the nucleic acid inhibitor molecule. Typically, the phosphate is added to a monomer prior to oligonucleotide synthesis. In other embodiments, 5′-phosphorylation is accomplished naturally after a nucleic acid inhibitor molecule is introduced into the cytosol, for example, by a cytosolic Clp1 kinase. In some embodiments, the 5′-terminal phosphate is a phosphate group, such as 5′-monophosphate [(HO)₂(O)P—O-5′], 5′-diphosphate [(HO)₂(O)P—O—P(HO)(O)—O-5′] or a 5′-triphosphate[(HO)₂(O)P—O—(HO)(O)P—O—P(HO)(O)-0-5′].

The 5′-end of the nucleic acid inhibitor molecule can also be modified. For example, in some embodiments, the 5′-end of the nucleic acid inhibitor molecule is attached to a phosphoramidate [(HO)₂(O)P—NH-5′, (HO)(NH₂)(O)P—O-5′]. In certain embodiments, the 5′-terminal end of the nucleic acid inhibitor molecule is attached to a phosphate mimic. Suitable phosphate mimics include 5′-phosphonates, such as 5′-methylenephosphonate (5′-MP) and 5′-(E)-vinylphosphonate (5′-VP). Lima et al., Cell, 2012, 150-883-94; WO2014/130607. Other suitable phosphate mimics include 4-phosphate analogs that are bound to the 4′-carbon of the sugar moiety (e.g., a ribose or deoxyribose or analog thereof) of the 5′-terminal nucleotide of an oligonucleotide as described in International Publication No. WO 2018/045317, which is hereby incorporated by reference in its entirety. For example, in some embodiments, the 5′-end of the nucleic acid inhibitor molecule is attached to an oxymethylphosphonate, where the oxygen atom of the oxymethyl group is bound to the 4′-carbon of the sugar moiety or analog thereof. In other embodiments, the phosphate analog is a thiomethylphosphonate or an aminomethylphosphonate, where the sulfur atom of the thiomethyl group or the nitrogen atom of the aminomethyl group is bound to the 4′-carbon of the sugar moiety or analog thereof.

In certain embodiments, the nucleic acid inhibitor molecules include one or more deoxyribonucleotides. Typically, the nucleic acid inhibitor molecules contain fewer than 5 deoxyribonucleotides. In certain embodiments, the nucleic acid inhibitor molecules include one or more ribonucleotides. In certain embodiments, all of the nucleotides of the nucleic acid inhibitor molecule are ribonucleotides.

In certain embodiments, one or two nucleotides of a nucleic acid inhibitor molecule are reversibly modified with a glutathione-sensitive moiety. Typically, the glutathione-sensitive moiety is located at the 2′-carbon of the sugar moiety and comprises a sulfonyl group. In certain embodiment, the glutathione-sensitive moiety is compatible with phosphoramidite oligonucleotide synthesis methods, as described, for example, in International Publication No. WO 2018/045317, which is hereby incorporated by reference in its entirety. In certain embodiments, more than two nucleotides of a nucleic acid inhibitor molecule are reversibly modified with a glutathione-sensitive moiety. In certain embodiments, most of the nucleotides are reversibly modified with a glutathione-sensitive moiety. In certain embodiments, all or substantially all the nucleotides of a nucleic acid inhibitor molecule are reversibly modified with a glutathione-sensitive moiety.

The at least one glutathione-sensitive moiety is typically located at the 5′- or 3′-terminal nucleotide of a single-stranded nucleic acid inhibitor molecule or the 5′- or 3′-terminal nucleotide of the passenger strand or the guide strand of a double-stranded nucleic acid inhibitor molecule. However, the at least one glutathione-sensitive moiety may be located at any nucleotide of interest in the nucleic acid inhibitor molecule.

In certain embodiments, a nucleic acid inhibitor molecule is fully modified, wherein every nucleotide of the fully modified nucleic acid inhibitor molecule is modified. In certain embodiments, the fully modified nucleic acid inhibitor molecule does not contain a reversible modification. In some embodiments, at least one, such as at least two, three, four, five, six, seven, eight, nine, 10, 11, 12, 13, 14, 15, 16, 17, 18, 19, or 20 nucleotides of a single stranded nucleic acid inhibitor molecule or the guide strand or passenger strand of a double stranded nucleic acid inhibitor molecule are modified.

In certain embodiments, the fully modified nucleic acid inhibitor molecule is modified with one or more reversible, glutathione-sensitive moieties. In certain embodiments, substantially all of the nucleotides of a nucleic acid inhibitor molecule are modified. In certain embodiments, more than half of the nucleotides of a nucleic acid inhibitor molecule are modified with a chemical modification other than a reversible modification. In certain embodiments, less than half of the nucleotides of a nucleic acid inhibitor molecule are modified with a chemical modification other than a reversible modification. Modifications can occur in groups on the nucleic acid inhibitor molecule or different modified nucleotides can be interspersed.

In certain embodiments of the nucleic acid inhibitor molecule, from one to every nucleotide is modified at the 2′-carbon. In certain embodiments, the nucleic acid inhibitor molecule (or the sense strand and/or antisense strand thereof) is partially or fully modified with 2′-F, 2′-O-Me, and/or 2′-MOE. In certain embodiments, every nucleotide of the sense and antisense strands of the nucleic acid inhibitor is modified with 2′-F or 2′-O-Me. In certain embodiments of the nucleic acid inhibitor molecule, from one to every phosphorous atom is modified and from one to every nucleotide is modified at the 2′-carbon.

### KRAS Nucleic Acid Inhibitor Molecules

The term “KRAS” refers to nucleic acid sequences encoding a KRas protein, peptide, or polypeptide (e.g., KRAS transcripts, such as the sequences of KRAS Genbank Accession Nos. NM_033360.2 and NM_004985.3). In certain embodiments, the term “KRAS” is also meant to include other KRAS-encoding sequences, such as other KRAS isoforms, mutant KRAS genes, splice variants of KRAS genes, and KRAS gene polymorphisms. The KRAS nucleic acid inhibitor molecules described herein can be designed to hybridize to any KRAS target sequence of interest, including those disclosed in as well as those referred to in U.S. Published Pat. Nos. 8,372,816; 8,513,207; 9,200,284; and 9,809,819 and U.S. Published Patent Application No. 2018/0044680, all of which are incorporated by reference herein. The term “Kras” is used to refer to the polypeptide gene product of a KRAS gene/transript, e.g., a Kras protein, peptide, or polypeptide, such as those encoded by KRAS Genbank Accession Nos. NM_033360.2 and NM_004985.3.

As used herein, a “KRAS-associated disease or disorder” refers to a disease or disorder known in the art to be associated with altered KRAS expression, level, and/or activity. Notably, a “KRAS-associated disease or disorder” includes cancer and/or proliferative diseases, conditions, or disorders. A “KRAS-associated cancer” refers to a cancer refers to a cancer known in the art to be associated with altered KRAS expression, level, and/or activity.

In certain embodiments, DsiRNA-mediated inhibition of a KRAS target sequence is assessed. In such embodiments, KRAS RNA levels can be assessed by art-recognized methods (e.g., RT-PCR, Northern blot, expression array, etc.), optionally via comparison of KRAS levels in the presence of an KRAS nucleic acid inhibitor molecule as disclosed herein relative to the absence of such KRAS nucleic acid inhibitor molecule. In certain embodiments, KRAS levels in the presence of a KRAS nucleic acid inhibitor molecule are compared to those observed in the presence of vehicle alone, in the presence of a nucleic acid inhibitor molecule directed against an unrelated target RNA, or in the absence of any treatment. It is also recognized that levels of Kras protein can be assessed as indicative of KRAS RNA levels and/or the extent to which a nucleic acid inhibitor molecule inhibits KRAS expression, thus art-recognized methods of assessing KRAS protein levels (e.g., Western blot, immunoprecipitation, other antibody-based methods, etc.) can also be employed to examine the inhibitory effect of a nucleic acid inhibitor molecule. A KRAS nucleic acid inhibitor molecule as disclosed herein is deemed to possess “KRAS inhibitory activity” if a statistically-significant reduction in KRAS RNA or protein levels is seen when a KRAS nucleic acid inhibitor molecule as disclosed herein is administered to a system (e.g., cell-free in vitro system), cell, tissue or organism, as compared to an appropriate control. The distribution of experimental values and the number of replicate assays performed will tend to dictate the parameters of what levels of reduction in KRAS RNA or protein (either as a % or in absolute terms) is deemed statistically significant (as assessed by standard methods of determining statistical significance known in the art). However, in certain embodiments, “KRAS inhibitory activity” is defined based upon a % or absolute level of reduction in the level of KRAS in a system, cell, tissue or organism. For example, in certain embodiments, a KRAS nucleic acid inhibitor molecule disclosed herein is deemed to possess KRAS inhibitory activity if at least a 5% reduction or at least a 10% reduction in KRAS RNA is observed in the presence of the nucleic acid inhibitor molecule relative to KRAS levels seen for a suitable control. (For example, in vivo KRAS levels in a tissue and/or subject can, in certain embodiments, be deemed to be inhibited by a nucleic acid inhibitor molecule as disclosed herein if, e.g., a 5% or 10% reduction in KRAS levels is observed relative to a control.)

In certain other embodiments, a KRAS nucleic acid inhibitor molecule as disclosed herein is deemed to possess KRAS inhibitory activity if KRAS RNA levels are observed to be reduced by at least 15% relative to an appropriate control, by at least 20% relative to an appropriate control, by at least 25% relative to an appropriate control, by at least 30% relative to an appropriate control, by at least 35% relative to an appropriate control, by at least 40% relative to an appropriate control, by at least 45% relative to an appropriate control, by at least 50% relative to an appropriate control, by at least 55% relative to an appropriate control, by at least 60% relative to an appropriate control, by at least 65% relative to an appropriate control, by at least 70% relative to an appropriate control, by at least 75% relative to an appropriate control, by at least 80% relative to an appropriate control, by at least 85% relative to an appropriate control, by at least 90% relative to an appropriate control, by at least 95% relative to an appropriate control, by at least 96% relative to an appropriate control, by at least 97% relative to an appropriate control, by at least 98% relative to an appropriate control or by at least 99% relative to an appropriate control. In some embodiments, complete inhibition of KRAS is required for a KRAS nucleic acid inhibitor molecule to be deemed to possess KRAS inhibitory activity. In certain models (e.g., cell culture), a KRAS nucleic acid inhibitor molecule is deemed to possess KRAS inhibitory activity if at least a 40% reduction in KRAS levels is observed relative to a suitable control. In certain embodiments, a KRAS nucleic acid inhibitor molecule is deemed to possess KRAS inhibitory activity if at least a 50% reduction in KRAS levels is observed relative to a suitable control. In certain other embodiments, a KRAS nucleic acid inhibitor molecule is deemed to possess KRAS inhibitory activity if at least an 80% reduction in KRAS levels is observed relative to a suitable control.

KRAS inhibitory activity can also be evaluated over time (duration) and over concentration ranges (potency), with assessment of what constitutes a nucleic acid inhibitor molecule possessing KRAS inhibitory activity adjusted in accordance with concentrations administered and duration of time following administration. Thus, in certain embodiments, a KRAS nucleic acid inhibitor molecule as disclosed herein is deemed to possess KRAS inhibitory activity if at least a 50% reduction in KRAS activity is observed at a duration of time of 2 hours, 5 hours, 10 hours, 1 day, 2 days, 3 days, 4 days, 5 days, 6 days, 7 days, 8 days, 9 days, 10 days or more after administration is observed/persists. In additional embodiments, a KRAS nucleic acid inhibitor molecule as disclosed herein is deemed to be a potent KRAS inhibitory agent if KRAS inhibitory activity (e.g., in certain embodiments, at least a 40% inhibition of KRAS or at least a 50% inhibition of KRAS) is observed at a concentration of 1 nM or less, 500 pM or less, 200 pM or less, 100 pM or less, 50 pM or less, 20 pM or less, 10 pM or less, 5 pM or less, 2 pM or less or even 1 pM or less in the environment of a cell.

Suitable nucleic acid inhibitor molecule compositions that contain two separate oligonucleotides can be chemically linked outside their annealing region by chemical linking groups. Many suitable chemical linking groups are known in the art and can be used. Suitable groups will not block Dicer activity on the nucleic acid inhibitor molecule and will not interfere with the directed destruction of the RNA transcribed from the target gene. Alternatively, the two separate oligonucleotides can be linked by a third oligonucleotide such that a hairpin structure is produced upon annealing of the two oligonucleotides making up the nucleic acid inhibitor molecule composition. The hairpin structure will not block Dicer activity on the nucleic acid inhibitor molecule and will not interfere with the directed destruction of the target RNA.

In certain embodiments, the KRAS nucleic acid inhibitor molecule is KRAS-194. In certain embodiments, the sense strand of the KRAS nucleic acid inhibitor molecule comprises or consists of the sequence of SEQ ID NO: 1 (5′-GGCCUGCUGAAAAUGACUGAAUATA-3′). In certain embodiments, the antisense strand of the KRAS nucleic acid inhibitor molecule comprises or consists of the sequence of SEQ ID NO: 2 (3′-CUCCGGACGACUUUUACUGACUUAUAU-5′). In certain embodiments, the sense strand of the KRAS nucleic acid inhibitor molecule comprises or consists of the sequence of SEQ ID NO: 1, and the antisense strand comprises or consists of the sequence of SEQ ID NO: 2.

In certain embodiments, the KRAS nucleic acid inhibitor molecule is KRAS-465. In certain embodiments, the sense strand of the KRAS nucleic acid inhibitor molecule comprises or consists of the sequence of SEQ ID NO: 3 (5′-CUAAAUCAUUUGAAGAUAUUCACCA-3′). In certain embodiments, the antisense strand of the KRAS nucleic acid inhibitor molecule comprises or consists of the sequence of SEQ ID NO: 4 (3′-AUGAUUUAGUAAACUUCUAUAAGUGGU-5′). In certain embodiments, the sense strand of the KRAS nucleic acid inhibitor molecule comprises or consists of the sequence of SEQ ID NO: 3, and the antisense strand comprises or consists of the sequence of SEQ ID NO: 4.

In certain embodiments, the KRAS nucleic acid inhibitor molecule is KRAS-446. In certain embodiments, the sense strand of the KRAS nucleic acid inhibitor molecule comprises or consists of the sequence of SEQ ID NO: 5 (5′-GUAUUUGCCAUAAAUAAUACUAAAT-3′). In certain embodiments, the antisense strand of the KRAS nucleic acid inhibitor molecule comprises or consists of the sequence of SEQ ID NO: 6 (3′-CACAUAAACGGUAUUUAUUAUGAUUUA-5′). In certain embodiments, the sense strand of the KRAS nucleic acid inhibitor molecule comprises or consists of the sequence of SEQ ID NO: 5, and the antisense strand comprises or consists of the sequence of SEQ ID NO: 6.

In certain embodiments, the KRAS nucleic acid inhibitor molecule is KRAS-194T. In certain embodiments, the sense strand of the KRAS nucleic acid inhibitor molecule comprises or consists of the sequence of SEQ ID NO: 7 (5′-GGCCUGCUGAAAAUGACUGAGCAGCCGAAAGGCUGC-3′). In certain embodiments, the antisense strand of the KRAS nucleic acid inhibitor molecule comprises or consists of the sequence of SEQ ID NO: 8 (3′-CUCCGGACGACUUUUACUGACU-5′). In certain embodiments, the sense strand of the KRAS nucleic acid inhibitor molecule comprises or consists of the sequence of SEQ ID NO: 7, and the antisense strand comprises or consists of the sequence of SEQ ID NO: 8. In certain embodiments (U/GG format), the sense strand comprises or consists of SEQ ID NO: 7 and the antisense strand comprises or consists of SEQ ID NO: 17 (3′-GGCCGGACGACUUUUACUGACU-5′).

In certain embodiments, the KRAS nucleic acid inhibitor molecule is KRAS-465T. In certain embodiments, the sense strand of the KRAS nucleic acid inhibitor molecule comprises or consists of the sequence of SEQ ID NO: 9 (5′-CUAAAUCAUUUGAAGAUAUUGCAGCCGAAAGGCUGC-3′). In certain embodiments, the antisense strand of the KRAS nucleic acid inhibitor molecule comprises or consists of the sequence of SEQ ID NO: 10 (3′-AUGAUUUAGUAAACUUCUAUAA-5′). In certain embodiments, the sense strand of the KRAS nucleic acid inhibitor molecule comprises or consists of the sequence of SEQ ID NO: 9, and the antisense strand comprises or consists of the sequence of SEQ ID NO: 10. In certain embodiments (U/GG format), the sense strand comprises or consists of SEQ ID NO: 13 (5′-CUAAAUCAUUUGAAGAUAUAGCAGCCGAAAGGCUGC-3′). In certain embodiments (U/GG format), the antisense strand comprises or consists of SEQ ID NO: 18 (3′-GGGAUUUAGUAAACUUCUAUAU-5′). In certain embodiments (U/GG format), the sense strand comprises or consists of SEQ ID NO: 13, and the antisense strand comprises or consists of SEQ ID NO: 18.

In certain embodiments, the KRAS nucleic acid inhibitor molecule is KRAS-446T. In certain embodiments, the sense strand of the KRAS nucleic acid inhibitor molecule comprises or consists of the sequence of SEQ ID NO: 11 (5′-GUAUUUGCCAUAAAUAAUACGCAGCCGAAAGGCUGC-3′). In certain embodiments, the antisense strand of the KRAS nucleic acid inhibitor molecule comprises or consists of the sequence of SEQ ID NO: 12 (3′-CACAUAAACGGUAUUUAUUAUG-5′). In certain embodiments, the sense strand of the KRAS nucleic acid inhibitor molecule comprises or consists of the sequence of SEQ ID NO: 11, and the antisense strand comprises or consists of the sequence of SEQ ID NO: 12. In certain embodiments (U/GG format), the sense strand comprises or consists of SEQ ID NO: (5′-GUAUUUGCCAUAAAUAAUAAGCAGCCGAAAGGCUGC-3′). In certain embodiments (U/GG format), the antisense strand comprises or consists of SEQ ID NO: 19 (3′-GGCAUAAACGGUAUUUAUUAUU-5′). In certain embodiments (U/GG format), the sense strand comprises or consists of SEQ ID NO: 15 and the antisense strand comprises or consists of SEQ ID NO: 19.

In certain embodiments, the KRAS nucleic acid inhibitor molecule is KRAS-465T/MOP. In certain embodiments, the sense strand of the KRAS nucleic acid inhibitor molecule comprises or consists of the sequence of SEQ ID NO: 13 (5′-CUAAAUCAUUUGAAGAUAUAGCAGCCGAAAGGCUGC-3′). In certain embodiments, the antisense strand of the KRAS nucleic acid inhibitor molecule comprises or consists of the sequence of SEQ ID NO: 14 (3′-GGGAUUUAGUAAACUUCUAUAU-5′, wherein underlining indicates a 4′-oxymethylphosphonate modification). In certain embodiments, the sense strand of the KRAS nucleic acid inhibitor molecule comprises or consists of the sequence of SEQ ID NO: 13, and the antisense strand comprises or consists of the sequence of SEQ ID NO: 14.

In certain embodiments, the KRAS nucleic acid inhibitor molecule is KRAS-446T/MOP. In certain embodiments, the sense strand of the KRAS nucleic acid inhibitor molecule comprises or consists of the sequence of SEQ ID NO: 15 (5′-GUAUUUGCCAUAAAUAAUAAGCAGCCGAAAGGCUGC-3′). In certain embodiments, the antisense strand of the KRAS nucleic acid inhibitor molecule comprises or consists of the sequence of SEQ ID NO: 16 (3′-GGCAUAAACGGUAUUUAUUAUU-5′, wherein underlining indicates a 4′-oxymethylphosphonate modification). In certain embodiments, the sense strand of the KRAS nucleic acid inhibitor molecule comprises or consists of the sequence of SEQ ID NO: 15, and the antisense strand comprises or consists of the sequence of SEQ ID NO: 16.

### MEK Inhibitors

As described herein, the term “MEK” refers to the mitogen-activated protein kinase kinase enzymes MEK1 and/or MEK2. MEK is also known as MAP2K and MAPKK. MEK is a member of the RAS/RAF/MEK/ERK signaling cascade that is activated in certain cancers, such as melanoma. The pathway is activated through the binding of a number of growth factors and cytokines to receptors on the cell surface, which activate receptor tyrosine kinases. Activation of the receptor tyrosine kinases results in activation of RAS, which then recruits RAF, which is in turn activated by multiple phosphorylation events.

Activated RAF phosphorylates and activates MEK kinase, which in turn phosphorylates and activates ERK kinase (also known as mitogen-activated protein kinase “MAPK”). The phosphorylated ERK can then translocate to the nucleus, where it phosphorylates and activates directly or indirectly various transcription factors, such as c-Myc and CREB. This process leads to altered gene transcription of genes that are important for cellular growth and proliferation.

As links in the RAS/RAF/4EK/ERK signaling cascade, MEK1 and MEK2 play roles in tumorigenesis, cell proliferation, and inhibition of apoptosis. Although MEK1/2 are themselves rarely mutated, constitutively active MEK has been found in more than 30% of primary tumor cell lines tested. One of the ways of halting this cascade is the inhibition of MEK. When MEK is inhibited, cell proliferation is blocked, and apoptosis is induced. Inhibition of MEK has, therefore, been an attractive target for development of pharmaceutical therapies.

MEK inhibitors include, but are not limited to, trametinib (GSK1120212), selumetinib, binimetinib (MEK162), cobimetinib (XL518), refametinib (BAY 86-9766), pimasertib, PD-325901, R05068760, CI-1040 (PD035901), AZD8330 (ARRY-424704), RO4987655 (CH4987655), RO5126766, WX-554, E6201, and TAK-733. In one embodiment, the MEK inhibitor is trametinib.

Trametinib is a small molecule kinase inhibitor and is approved for use as a single agent or in combination with dabrafenib for the treatment of subjects with unresectable or metastatic melanoma with a V600E or V600K mutation in the BRAF gene. BRAF encodes a serine/threonine kinase called B-Raf that is involved in intracellular signaling.

### Immunotherapeutic Agents

Various methods and compositions disclosed herein relate to combination therapy with a KRAS nucleic acid inhibitor molecule and an immunotherapeutic agent. Administering the KRAS nucleic acid inhibitor molecule can render certain tumors that are not responsive to immunotherapy susceptible to immunotherapy.

Immunotherapy refers to methods of enhancing an immune response. Typically, in the methods disclosed herein an anti-tumor immune response is enhanced. In certain embodiments, immunotherapy refers to methods of enhancing a T cell response against a tumor or cancer.

In certain embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory immune checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CD8, CTLA4 or PD-1. In other embodiments, the inhibitory immune checkpoint molecule is a ligand for PD-1, such as CD274 (PD-L1) or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these inhibitory immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody and preferably is a monoclonal antibody. In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In certain embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In certain embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

### Pharmaceutical Compositions

The present disclosure provides pharmaceutical compositions comprising a KRAS nucleic acid inhibitor molecule and a pharmaceutically acceptable excipient. In certain embodiments, the pharmaceutical composition comprising the KRAS nucleic acid inhibitor molecule and the pharmaceutically acceptable excipient further comprises an MEK inhibitor. In certain embodiments, the pharmaceutical composition comprising the KRAS nucleic acid inhibitor molecule and the pharmaceutically acceptable excipient further comprises an immunotherapy agent.

The pharmaceutically acceptable excipients useful in this disclosure are conventional. Remington's Pharmaceutical Sciences, by E. W. Martin, Mack Publishing Co., Easton, Pa., 15^{th} Edition (1975), describes compositions and formulations suitable for pharmaceutical delivery of one or more therapeutic compositions, including vaccines, and additional pharmaceutical agents. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In general, the nature of the excipient will depend on the particular mode of administration being employed. For instance, parenteral formulations usually comprise injectable fluids that include pharmaceutically and physiologically acceptable fluids such as water, physiological saline, balanced salt solutions, buffers, aqueous dextrose, glycerol or the like as a vehicle. For solid compositions (for example, powder, pill, tablet, or capsule forms), conventional non-toxic solid excipients can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, a surface active agent, preservatives, and pH buffering agents and the like, for example sodium acetate or sorbitan monolaurate. In certain embodiments, the pharmaceutically acceptable excipient is non-naturally occurring.

The pharmaceutical composition according to certain embodiments disclosed herein may comprise at least one ingredient, which may belong to the same or different categories of excipients, including at least one disintegrant, at least one diluent, and/or at least one binder.

Typical non-limiting examples of the at least one disintegrant that may be added to the pharmaceutical composition according to embodiments disclosed herein, include, but are not limited to, povidone, crospovidone, carboxymethylcellulose, methylcellulose, alginic acid, croscarmellose sodium, sodium starch glycolate, starch, formaldehyde-casein, and combinations thereof.

Typical non-limiting examples of the at least one diluents that may be added to the pharmaceutical composition according to embodiments disclosed herein, include, but are not limited to, maltose, maltodextrin, lactose, fructose, dextrin, microcrystalline cellulose, pregelatinized starch, sorbitol, sucrose, silicified microcrystalline cellulose, powdered cellulose, dextrates, mannitol, calcium phosphate, and combinations thereof.

Typical non-limiting examples of the at least one binder that may be added to the pharmaceutical composition according to embodiments disclosed herein, include, but are not limited to, acacia, dextrin, starch, povidone, carboxymethylcellulose, guar gum, glucose, hydroxypropyl methylcellulose, methylcellulose, polymethacrylates, maltodextrin, hydroxyethyl cellulose, and combinations thereof.

Suitable preparation forms for the pharmaceutical compositions disclosed herein include, for example, tablets, capsules, soft capsules, granules, powders, suspensions, aerosols, emulsions, microemulsions, nanoemulsions, unit dosage forms, rings, films, suppositories, solutions, creams, syrups, transdermal patches, ointments, or gels.

The KRAS nucleic acid inhibitor molecule may be admixed, encapsulated, conjugated or otherwise associated with other molecules, molecule structures or mixtures of compounds, including, for example, liposomes and lipids such as those disclosed in U.S. Pat. Nos. 6,815,432, 6,586,410, 6,858,225, 7,811,602, 7,244,448 and 8,158,601; polymeric materials such as those disclosed in U.S. Pat. Nos. 6,835,393, 7,374,778, 7,737,108, 7,718,193, 8,137,695 and U.S. Published Patent Application Nos. 2011/0143434, 2011/0129921, 2011/0123636, 2011/0143435, 2011/0142951, 2012/0021514, 2011/0281934, 2011/0286957 and 2008/0152661; capsids, capsoids, or receptor targeted molecules for assisting in uptake, distribution or absorption.

In certain embodiments, the nucleic acid inhibitor molecules are formulated in a lipid nanoparticle (LNP). Lipid-nucleic acid nanoparticles typically form spontaneously upon mixing lipids with nucleic acid to form a complex. Depending on the desired particle size distribution, the resultant nanoparticle mixture can be optionally extruded through a polycarbonate membrane (e.g., 100 nm cut-off) using, for example, a thermobarrel extruder, such as Lipex Extruder (Northern Lipids, Inc). To prepare a lipid nanoparticle for therapeutic use, it may desirable to remove solvent (e.g., ethanol) used to form the nanoparticle and/or exchange buffer, which can be accomplished by, for example, dialysis or tangential flow filtration. Methods of making lipid nanoparticles containing nucleic acid inhibitor molecules are known in the art, as disclosed, for example in U.S. Published Patent Application Nos. 2015/0374842 and 2014/0107178.

In certain embodiments, the LNP comprises a liposome comprising a cationic liposome and a pegylated lipid. The LNP can further comprise one or more envelope lipids, such as a cationic lipid, a structural lipid, a sterol, a pegylated lipid, or mixtures thereof.

Cationic lipids for use in LNPs are known in the art, as discussed for example in U.S. Published Patent Application Nos. 2015/0374842 and 2014/0107178. Typically, the cationic lipid is a lipid having a net positive charge at physiological pH. In certain embodiments, the cationic liposome is DODMA, DOTMA, DL-048, or DL-103. In certain embodiments the structural lipid is DSPC, DPPC or DOPC. In certain embodiments, the sterol is cholesterol. In certain embodiments, the pegylated lipid is DMPE-PEG, DSPE-PEG, DSG-PEG, DMPE-PEG2K, DSPE-PEG2K, DSG-PEG2K, or DSG-MPEG. In one embodiment, the cationic lipid is DL-048, the pegylated lipid is DSG-MPEG and the one or more envelope lipids are DL-103, DSPC, cholesterol, and DSPE-MPEG.

In certain embodiments, the KRAS nucleic acid inhibitor molecule is covalently conjugated to a ligand that directs delivery of the oligonucleotide to a tissue of interest. Many such ligands have been explored. See, e.g., Winkler, Ther. Deliv. 4(7): 791-809 (2013). For example, the KRAS nucleic acid inhibitor molecule can be conjugated to one or more sugar ligand moieties (e.g., N-acetylgalactosamine (GalNAc)) to direct uptake of the oligonucleotide into the liver. See, e.g., U.S. Pat. Nos. 5,994,517; 5,574,142; WO 2016/100401. Typically, the KRAS nucleic acid inhibitor molecule is conjugated to three or four sugar ligand moieties (e.g., GalNAc). Other ligands that can be used include, but are not limited to, mannose-6-phosphate, cholesterol, folate, transferrin, and galactose (for other specific exemplary ligands see, e.g., WO 2012/089352). Typically, when an oligonucleotide is conjugated to a ligand, the oligonucleotide is administered as a naked oligonucleotide, wherein the oligonucleotide is not also formulated in an LNP or other protective coating. In certain embodiments, each nucleotide within the naked oligonucleotide is modified at the 2′-position of the sugar moiety, typically with 2′-F, 2′-OMe, and/or 2′-MOE.

These pharmaceutical compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above mentioned agent or agents, such as in a sealed package of tablets or capsules. The composition in solid form can also be packaged in a container for a flexible quantity, such as in a squeezable tube designed for a topically applicable cream or ointment.

In certain embodiments, the pharmaceutical compositions described herein are for use in treating a KRAS-associated disease or disorder, such as KRAS-associated cancer. In certain embodiments, the pharmaceutical composition for use in treating a KRAS-associated disease or disorder comprises a KRAS nucleic acid inhibitor molecule, wherein the composition is administered in combination with a MEK inhibitor (e.g., trametinib). In certain embodiments, the pharmaceutical composition for use in treating a KRAS-associated disease or disorder comprises a KRAS nucleic acid inhibitor molecule, wherein the composition is administered in combination with an immunotherapeutic agent. In other embodiments, the pharmaceutical composition for use in treating a KRAS-associated disease or disorder comprises a KRAS nucleic acid inhibitor molecule, wherein the composition is administered in combination with a different chemotherapeutic agent, such as a TGF-3 inhibitor molecule or a CSF-1 antibody. In certain embodiments, the KRAS-associated disease or disorder is cancer, such as pancreatic cancer, colorectal cancer, hepatocellular carcinoma, or melanoma. In certain embodiments, the KRAS-associated cancer has metastasized. In certain embodiments, the KRAS-associated cancer is pancreatic cancer.

### Dosage Forms

The pharmaceutical compositions disclosed herein may be formulated with conventional excipients for any intended route of administration.

Typically, the pharmaceutical compositions of the present disclosure that contain a KRAS nucleic acid inhibitor molecule are formulated in liquid form for parenteral administration, for example, by subcutaneous, intramuscular, intravenous or epidural injection. Typically, the pharmaceutical compositions that contain an immunotherapeutic agent, such as an antagonist of an inhibitory immune checkpoint molecule (e.g., one or more of an anti-CTLA-4, anti-PD-1, or anti-PD-L1 antibody) or an agonist of a co-stimulatory checkpoint molecule are formulated in liquid form for parenteral administration, for example, by subcutaneous, intramuscular, intravenous or epidural injection.

Dosage forms suitable for parenteral administration typically include one or more suitable vehicles for parenteral administration including, by way of example, sterile aqueous solutions, saline, low molecular weight alcohols such as propylene glycol, polyethylene glycol, vegetable oils, gelatin, fatty acid esters such as ethyl oleate, and the like. The parenteral formulations may contain sugars, alcohols, antioxidants, buffers, bacteriostats, solutes which render the formulation isotonic with the blood of the intended recipient or suspending or thickening agents. Proper fluidity can be maintained, for example, by the use of surfactants. Liquid formulations can be lyophilized and stored for later use upon reconstitution with a sterile injectable solution.

The pharmaceutical compositions may also be formulated for other routes of administration including topical or transdermal administration, rectal or vaginal administration, ocular administration, nasal administration, buccal administration, or sublingual administration.

### Methods of Administration/Treatment

Typically, the nucleic acid inhibitor molecules of the invention are administered intravenously or subcutaneously. However, the pharmaceutical compositions disclosed herein may also be administered by any method known in the art, including, for example, oral, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like. Administration may also be via injection, for example, intraperitoneally, intramuscularly, intradermally, intraorbitally, intracapsularly, intraspinally, intrasternally, or the like.

The therapeutically-effective amount of the compounds disclosed herein may depend on the route of administration and the physical characteristics of the patient, such as general state, weight, diet, and other medications. As used herein, a therapeutically-effective amount means an amount of compound or compounds effective to prevent, alleviate or ameliorate disease or condition symptoms of the subject being treated. Determination of a therapeutically-effective amount is well within the capability of those skilled in the art and generally range from about 0.5 mg to about 3000 mg of the small molecule agent or agents per dose per patient.

In one aspect, the pharmaceutical compositions disclosed herein may be useful for the treatment or prevention of symptoms related to a KRAS-associated disease or disorder. One embodiment is directed to a method of treating a KRAS-associated disease or disorder, comprising administering to a subject a therapeutically effective amount of a KRAS nucleic acid inhibitor molecule. One embodiment is directed to a method of treating a KRAS-associated disease or disorder, comprising administering to a subject a therapeutically effective amount of a KRAS nucleic acid inhibitor molecule and a therapeutically effective amount of an MEK inhibitor. One embodiment is directed to a method of treating a KRAS-associated disease or disorder, comprising administering to a subject a therapeutically effective amount of a KRAS nucleic acid inhibitor molecule and a therapeutically effective amount of an immunotherapeutic agent. Another embodiment is directed to a method of treating a KRAS-associated disease or disorder, comprising administering to a subject a therapeutically effective amount of a KRAS nucleic acid inhibitor molecule and a therapeutically effective amount of a chemotherapeutic agent, such as a TGF-β inhibitor molecule or a CSF-1 antibody.

Typically, the nucleic acid inhibitor molecule is administered separately from, and on a different schedule than, a small molecule therapeutic that is in combination with the nucleic acid inhibitor molecule, such as an MEK inhibitor. For example, when used as a single agent, trametinib is currently prescribed as a daily oral dose (typically about 1-2 mg/day). The nucleic acid inhibitor molecule, on the other hand, is likely to be administered through an intravenous or subcutaneous route with doses given once a week, once each two weeks, once a month, once every three months, twice a year, etc. The subject may already be taking the small molecule therapeutic at the initiation of the administration of the nucleic acid inhibitor molecule. In other embodiments, the subject may begin administration of both the small molecule therapeutic and the nucleic acid inhibitor molecule at about the same time. In other embodiments, the subject may begin taking the small molecule therapeutic after the initiation of administration of the nucleic acid inhibitor molecule. In certain embodiments, the subject may be administered the nucleic acid inhibitor molecule after the subject begins taking the small molecule therapeutic, such as after the subject has discontinued taking the small molecule therapeutics.

Additionally, the nucleic acid inhibitor molecule may be administered separately from, and on a different schedule than, an immunotherapeutic agent. For example, when used as a single agent, ipilimumab (anti-CTLA-4 antibody) is administered intravenously over 90 minutes at a recommended dose of 3 mg/kg every 3 weeks for a total of 4 doses. Similarly, when used as a single agent, nivolumab (anti-PD-1 antibody), is administered intravenously at a recommended dose of 240 mg (or 3 mg/kg) over 60 minutes every 2 weeks. When nivolumab is administered in combination with ipilimumab, the recommended dose of nivolumab is 1 mg/kg administered intravenously over 60 minutes, followed by ipilimumab on the same day at a recommended dose of 3 mg/kg every 3 weeks for a total of 4 doses, and then nivolumab at a recommended dose of 240 mg every 2 weeks. When pembrolizumab is used as a single agent, it is typically administered intravenously over 30 minutes at a recommended dosage of 200 mg every 3 weeks until disease progression, unacceptable toxicity, or up to 24 months without disease progression.

In certain embodiments for the methods of treatment disclosed herein, one pharmaceutical composition may comprise the KRAS nucleic acid inhibitor molecule and a separate pharmaceutical composition may comprise the MEK inhibitor.

In other embodiments, the KRAS nucleic acid inhibitor molecule may be administered simultaneously with the MEK inhibitor.

Accordingly, in certain embodiments for the methods of treatment disclosed herein, a single pharmaceutical composition may comprise both the KRAS nucleic acid inhibitor molecule and the MEK inhibitor and/or the immunotherapeutic agent. Thus, in one embodiment of the treatment methods disclosed herein, a single pharmaceutical composition is administered to the subject, wherein the single pharmaceutical composition comprises both the KRAS nucleic acid inhibitor molecule and the MEK inhibitor, such as trametinib.

In certain embodiments, the KRAS nucleic acid inhibitor molecule is administered at a dosage of 20 micrograms to 10 milligrams per kilogram body weight of the recipient per day, 100 micrograms to 5 milligrams per kilogram, 0.25 milligrams to 2.0 milligrams per kilogram, or 0.5 to 2.0 milligrams per kilogram.

In certain embodiments, the KRAS nucleic acid inhibitor molecule is administered once daily, once weekly, once every two weeks, once monthly, once every two months, once a quarter, twice a year, or once yearly. In certain embodiments, the KRAS nucleic acid inhibitor molecule is administered once or twice every 2, 3, 4, 5, 6, or 7 days. The compositions (containing both agents or a single, individual agent) can be administered once monthly, once weekly, once daily (QD), once every other day, or divided into multiple monthly, weekly, or daily doses, such as twice daily, three times a day or once every two weeks. In certain embodiments, the compositions can be administered once a day for two, three, four, five, six, or at least seven days. Although the agents can be administered simultaneously, typically each agent will be administered on a different schedule, particularly if the agents are administered via different routes.

Alternatively, continuous intravenous infusion sufficient to maintain therapeutically effective concentrations in the blood are contemplated. The skilled artisan will appreciate that certain factors may influence the dosage and timing required to effectively treat a subject, including but not limited to the severity of the disease or disorder, previous treatments, the general health and/or age or weight of the subject, and other diseases present.

Treatment of a subject with a therapeutically effective amount of an agent can include a single treatment or, preferably, can include a series of treatments. In certain embodiments, the treatment schedule includes a first loading dosage or phase, which is typically a higher dosage or frequency, followed by a maintenance dosage or phase, which is typically a lower dosage or frequency than the loading dosage/phase. Typically, the treatment continues until disease progression or unacceptable toxicity occurs.

In certain embodiments, the KRAS nucleic acid inhibitor molecules can be inserted into expression constructs, e.g., viral vectors, retroviral vectors, expression cassettes, or plasmid viral vectors, e.g., using methods known in the art. Expression constructs can be delivered to a subject by, for example, inhalation, orally, intravenous injection, local administration (see U.S. Pat. No. 5,328,470) or by stereotactic injection (see e.g., Chen et al. (1994), Proc. Natl. Acad. Sci. USA, 91, 3054-3057).

The expression constructs may be constructs suitable for use in the appropriate expression system and include, but are not limited to retroviral vectors, linear expression cassettes, plasmids and viral or virally-derived vectors, as known in the art. Such expression constructs may include one or more inducible promoters, RNA Pol III promoter systems such as U6 snRNA promoters or Hi RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct, e.g., Tuschl (2002, Nature Biotechnol 20: 500-505).

One aspect is directed to methods of treating a KRAS-associated disease or disorder, comprising administering to a subject (preferably a human) a therapeutically effective amount of a KRAS nucleic acid inhibitor molecule, as described herein, and a therapeutically effective amount of an MEK inhibitor or an immunotherapeutic agent.

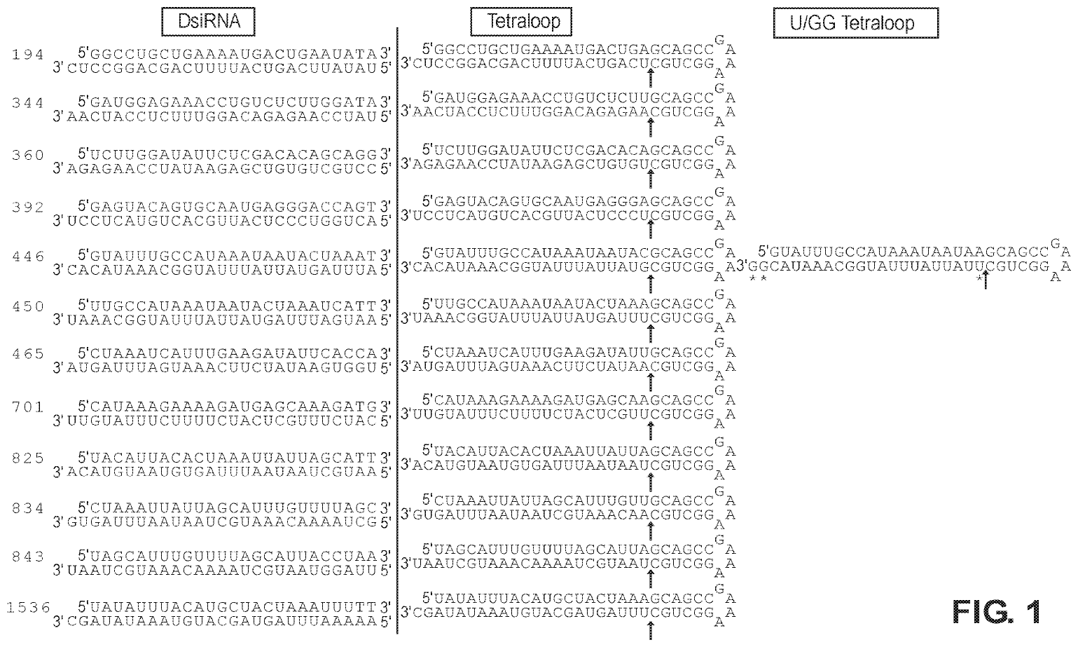

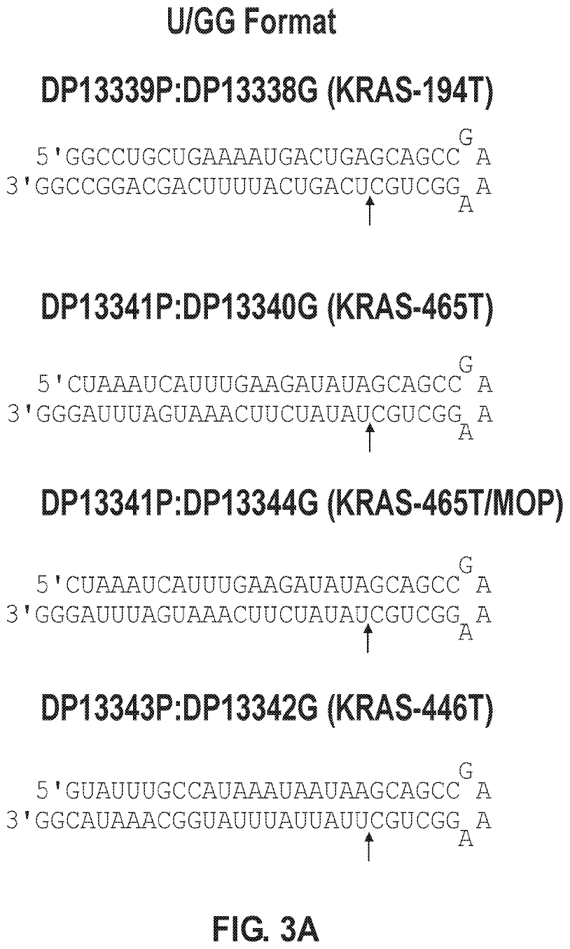

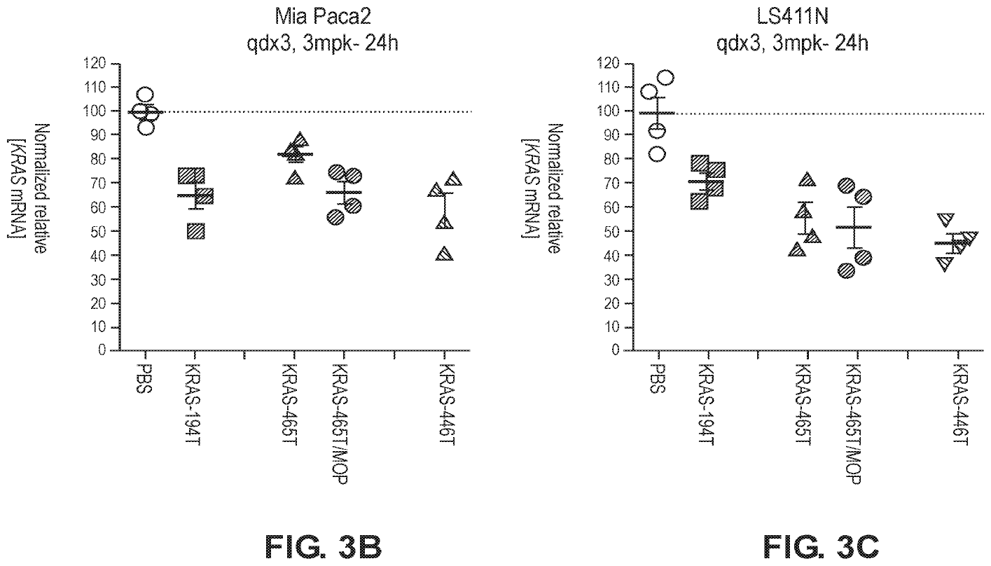

In one embodiment, the KRAS nucleic acid inhibitor molecule is a dsRNAi inhibitor molecule. In certain of those embodiments, the sense strand comprises or consists of the sequence of SEQ ID NO: 13 and/or the antisense strand comprises or consists of the sequence of SEQ ID NO: 14. In certain embodiments, the sense strand comprises or consists of SEQ ID NO: 13 and the antisense strand comprises or consists of SEQ ID NO: 14. In certain of those embodiments, the sense strand comprises or consists of the sequence of SEQ ID NO: 15 and/or the antisense strand comprises or consists of the sequence of SEQ ID NO: 16. In certain embodiments, the sense strand comprises or consists of SEQ ID NO: 15 and the antisense strand comprises or consists of SEQ ID NO: 16. In one embodiment, the KRAS nucleic acid inhibitor molecule comprises a tetraloop. In one embodiment the KRAS nucleic acid inhibitor molecule is formulated with a lipid nanoparticle. In one embodiment, the KRAS nucleic acid inhibitor molecule is administered intravenously. In certain embodiments, the sense strand comprises or consists of the sequence of one of SEQ ID NOs: 1, 3, 5, 7, 9, 11, 13, or 15. In certain embodiments, the antisense strand comprises or consists of the sequence of one of SEQ ID NOs: 2, 4, 6, 8, 10, 12, 14, or 16. Any of the DsiRNA or tetraloop structures of FIG. 1 or FIG. 3A can also be used in the methods described herein.

In one embodiment, the method of treatment comprises administering to a subject (preferably a human) a therapeutically effective amount of a KRAS nucleic acid inhibitor molecule and a therapeutically effective amount of an MEK inhibitor. In one embodiment, the MEK inhibitor is trametinib. In one embodiment, the trametinib is administered orally. In one embodiment, trametinib is administered at a dosage of about 1-2 mg daily or every other day. In one embodiment, trametinib is administered at a dosage of 2 mg daily.

In one embodiment, the MEK inhibitor is trametinib, which is administered orally, and the KRAS nucleic acid inhibitor molecule is a dsRNAi inhibitor molecule, wherein the region of complementarity between the sense strand and the antisense strand of the dsRNAi inhibitor molecule is between 15 and 40 nucleotides in length, including, for example, a double stranded nucleic acid having a sense strand and an antisense strand, wherein the sense strand comprises or consists of the sequence of SEQ ID NO: 13 and the antisense strand comprises of consists of the sequence of SEQ ID NO: 14. In certain embodiments, the region of complementarity between the sense strand and the antisense strand of the dsRNAi inhibitor molecule is about 20-35, such as about 25-35, about 20-26, about 25, or about 26 nucleotides in length. The KRAS dsRNAi inhibitor molecule can be formulated with a lipid nanoparticle and administered intravenously.

In certain embodiments of these treatment methods, the KRAS-associated disease or disorder is cancer, such as pancreatic cancer, colorectal cancer, hepatocellular carcinoma, or melanoma.

In certain embodiments of these treatment methods, the KRAS-associated cancer has metastasized. In certain embodiments, the KRAS-associated cancer is pancreatic cancer that has metastasized. In certain embodiments, the treatment reduces metastases in the subject. In certain embodiments, the treatment with the combination of a KRAS nucleic acid inhibitor molecule, such as a dsRNAi inhibitor molecule, and an MEK inhibitor, such as trametinib, increases survival of the subject beyond the average survival of patients with the cancer who receive treatment with either the KRAS nucleic acid inhibitor molecule or the MEK inhibitor (individually rather than in combination).

### EXAMPLES

### Example 1: KRAS Construct

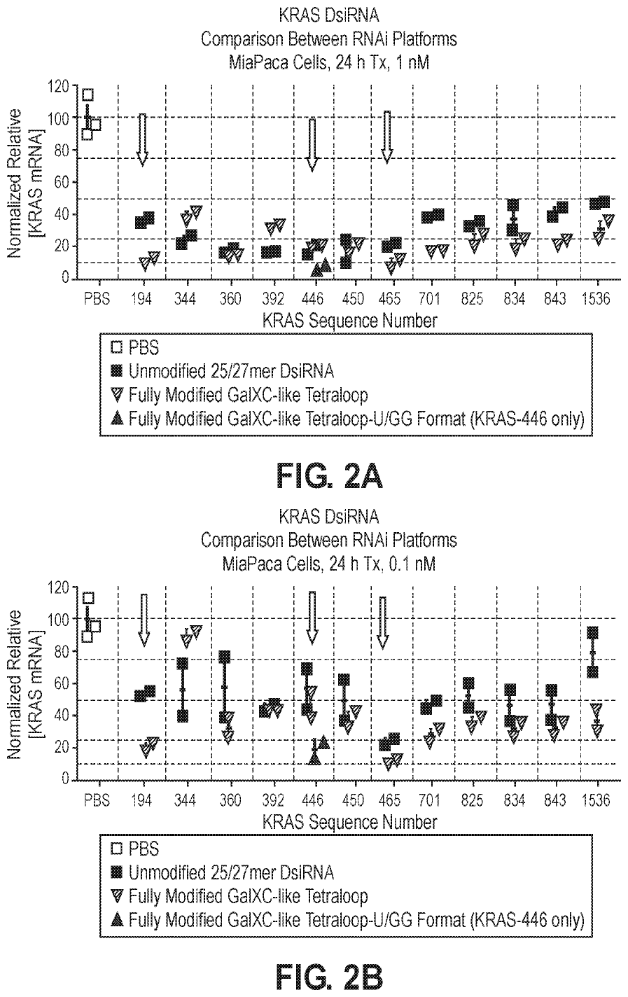

A nucleic acid inhibitor molecule that targets the KRAS gene (KRAS1) was constructed. First, several 25/27-mer KRAS DsiRNAs (with no modifications except for three methyls on the 3′ end of the guide strand) were selected. These constructs were then converted into a nicked-tetraloop format, using the U/GG convention as 22-mers, such that bases came off the guide strand starting from the 5′ end. See FIG. 1. These DsiRNAs were screened in human pancreatic carcinoma (MIA PaCa2) cells in vitro using lipofectamine at 1 nM and 0.1 nM concentrations of each construct to determine the potency. See FIGS. 2A and 2B.

The best three sequences (KRAS-194, KRAS-465, and KRAS-446 from this in vitro screen were then selected and constructed as tetraloops with a U/GG format (KRAS-194T, KRAS-465T, and KRAS-446T) and formulated in EnCore lipid nanoparticles (LNPs). See FIG. 3A.

In particular, the sense strand of KRAS-194 contains SEQ ID NO: 1 (5′-GGCCUGCUGAAAAUGACUGAAUATA-3′), and the antisense strand of KRAS-194 contains SEQ ID NO: 2 (3′-CUCCGGACGACUUUUACUGACUUAUAU-5′). The sense strand of KRAS-465 contains SEQ ID NO: 3 (5′-CUAAAUCAUUUGAAGAUAUUCACCA-3′), and the antisense strand of KRAS-465 contains SEQ ID NO: 4 (3′-AUGAUUUAGUAAACUUCUAUAAGUGGU-5′). The sense strand of KRAS-446 contains SEQ ID NO: 5 (5′-GUAUUUGCCAUAAAUAAUACUAAAT-3′), and the antisense strand of KRAS-446 contains SEQ ID NO: 6 (3′-CACAUAAACGGUAUUUAUUAUGAUUUA-5′).

In the U/GG format, the sense strand of KRAS-194T contains SEQ ID NO: 7 (5′-GGCCUGCUGAAAAUGACUGAGCAGCCGAAAGGCUGC-3′), and the antisense strand of KRAS-194T contains SEQ ID NO: 17 (3′-GGCCGGACGACUUUUACUGACU-5′). In the U/GG format, the sense strand of KRAS-465T contains SEQ ID NO: 13 (5′-CUAAAUCAUUUGAAGAUAUAGCAGCCGAAAGGCUGC-3′), and the antisense strand of KRAS-465T contains SEQ ID NO: 18 (3′-GGGAUUUAGUAAACUUCUAUAU-5′). In the U/GG format, the sense strand of KRAS-446T contains SEQ ID NO: 15 (5′-GUAUUUGCCAUAAAUAAUAAGCAGCCGAAAGGCUGC-3′), and the antisense strand of KRAS-446T contains SEQ ID NO: 19 (3′-GGCAUAAACGGUAUUUAUUAUU-5′).

The tetraloop sequences were tested in tumor models using MIA PaCa 2 and colon cancer LS411N cell lines. See FIGS. 3B and 3C. The best two from this screen (KRAS-465T and KRAS-446T) were then further modified to have a 4′-oxymethylphosphonate modification on the nucleotide at the 5′-end of the antisense strand (KRAS-465T/MOP and KRAS-446T/MOP). The sense strand of KRAS-465T/MOP contains SEQ ID NO: 13 (5′-CUAAAUCAUUUGAAGAUAUAGCAGCCGAAAGGCUGC-3′), and the antisense strand of KRAS-465T/MOP contains SEQ ID NO: 14 (3′-GGGAUUUAGUAAACUUCUAUAU-5′, wherein underlining indicates a 4′-oxymethylphosphonate modification). The sense strand of KRAS-446T/MOP contains SEQ ID NO: 15 (5′-GUAUUUGCCAUAAAUAAUAAGCAGCCGAAAGGCUGC-3′), and the antisense strand of KRAS-446T/MOP contains SEQ ID NO: 16 (3′-GGCAUAAACGGUAUUUAUUAUU-5′, wherein underlining indicates a 4′-oxymethylphosphonate modification).

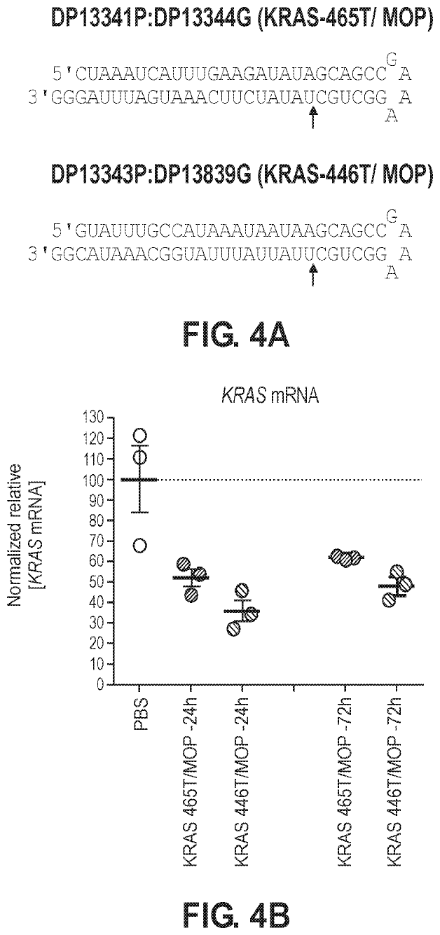

The two constructs were screened in LS411N tumors at 24 hour and 72 hour time points. See FIGS. 4A and 4B. KRAS-465T/MOP (or KRAS1) was selected for use in the tumor studies described below in Examples 2-8.

### Example 2: Methodology for Tumor Studies

6 to 8 week old immunocompetent or immunocompromised mice (C57BL/6/Nude) were injected subcutaneously with 2×10⁶ Pan02 (mouse pancreatic cell line) or 5×10⁶ Panc1 (human pancreatic cell line) tumor cells under the right shoulder. Tumor volume was measured every 2-3 days a week to monitor tumor growth. Dosing was initiated when the tumors reached about 200 mm³. For tumor growth inhibition studies, animals were randomized and assigned to different cohorts and subjected to dosing cycles. KRAS1-formulated LNP (“KRAS/LNP”) or Placebo (scrambled KRAS dsRNAi) formulated LNP was given intravenously via lateral tail vein at a total volume of 10 ml/kg. Immuno-modulatory agents (CSF1 antibody, TGF-β inhibitor, or checkpoint inhibitors) were given intraperitoneally or orally at a volume of 10 ml/kg. Trametinib (MEK inhibitor) was given orally at a total volume of 10 ml/kg.

Mouse pancreatic cell line Pan02 was obtained from NCI, and human pancreatic cell line Panc1 cells were obtained from ATCC (Manassas, Va.) and grown in RPMI/DMEM medium supplemented with 10% FBS. Pan02 is a murine PDAC cell line with KRAS G12D mutation. Panc1 is a human PDAC cell line with KRAS G1D mutation.

### Example 3: KRAS Nucleic Acid Inhibitor Molecule Treatment in Murine and Human PDAC with KRAS G12D Mutation

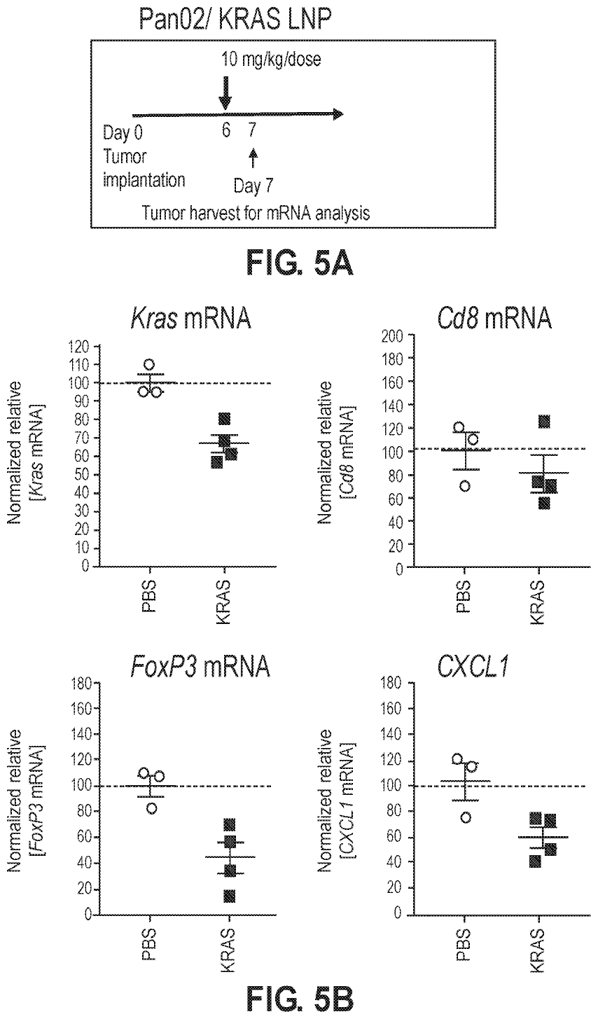

KRAS1 and Placebo were formulated in EnCore LNPs and used in the following studies. To evaluate if the LNP-formulated KRAS1 would effectively deliver the nucleic acid payload to pancreatic adenocarcinoma (PDAC) tumors, C57BL/6 mice were implanted with murine PDAC Pan02 tumors. At fourteen days post Pan02 tumor cell implantation, with the average tumor size of about 200 mm³, mice were sorted into two groups and were treated with either KRAS/LNP or Placebo/LNP at 10 mg/kg. See FIG. 5A. Twenty-four hours after the last dose, tumors were collected and analyzed by qPCR for mRNA levels of KRAS. Expression levels of the KRAS gene decreased about 40-50% as compared to control levels in tumors from mice treated with KRAS/LNP. See FIG. 5B. Likewise, expression levels of CD8, FoxP3, and CXCL1 all decreased. See FIG. 5B.

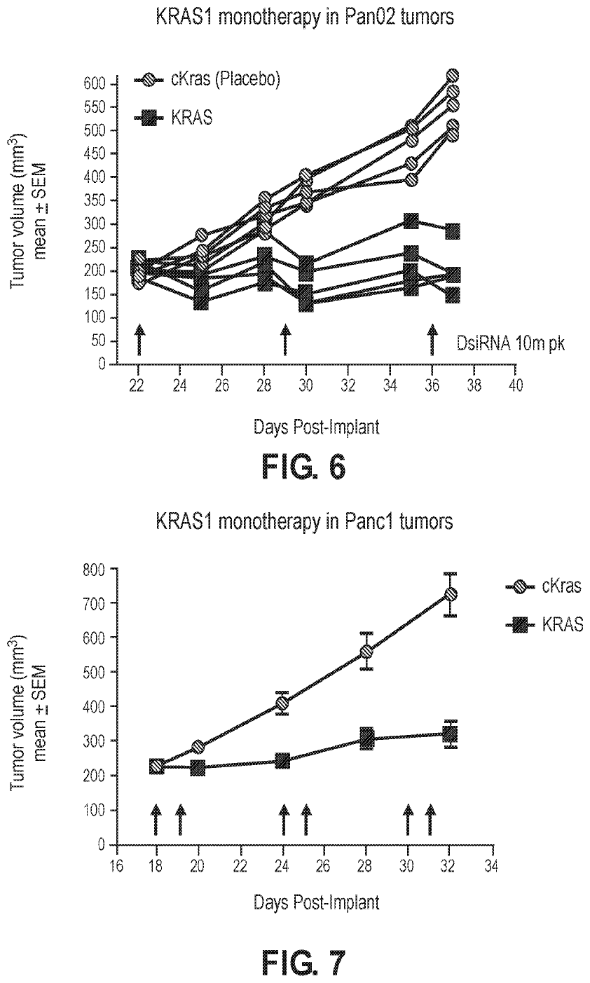

To see if the KRAS knockdown observed could be translated into growth inhibition, Pan02 tumors were implanted as described above, and when they reached the right sizes (e.g., about 200 mm³), they were sorted and treated with KRAS/LNP or Placebo/LNP (cKras) at 10 mpk once a week for 3 weeks, and the tumor growth was monitored. As shown in FIG. 6, complete growth inhibition was observed for the KRAS/LNP treated Pan02 tumors.

To see if KRAS/LNP would have the same effect in human tumors, human PDAC Panc1 cells were implanted in nude mice, and when they reached the average size of 200 mm³, they were sorted into 2 groups and treated at 5 mpk (qdx2, 5 mpk) with either KRAS/LNP or Placebo/LNP (cKras) over 3 weeks. Tumor growth was monitored, and as shown in FIG. 7, the Panc1 tumors, like the Pan02 tumors, also demonstrated complete growth inhibition, suggesting that about 40-50% KRAS knockdown may be sufficient to demonstrate complete tumor growth inhibition in KRAS-dependent pancreatic tumors.

### Example 4: KRAS Inhibition Leads to Modulation of Suppressive Molecules but not Stromal Activation Markers in Tumor Microenvironment of Murine Pancreatic Cancer

To see if a single KRAS/LNP treatment would lead to modulation of the tumor microenvironment, samples from the study described in Example 3 (FIGS. 5A-B) were analyzed for certain T-cell markers (CD8 and FoxP3) and chemokines (CXCL1). FoxP3 is a marker for immunosuppressive T cells (Tregs), which play an important role in regulating or suppressing other cells of the immune system. CXCL1 is a chemokine that actively recruits suppressive molecules such as Tregs and myeloid-derived suppressor cells to the tumor microenvironment. Interestingly, KRAS1-treated tumors had significantly decreased levels of both FoxP3 and CXCL1 mRNA in the tumor microenvironment. However, the CD8 levels were unchanged upon single treatment, suggesting that a single treatment of KRAS1 may not be enough to increase the T-cell infiltration into the suppressive tumor microenvironment of Pan02.

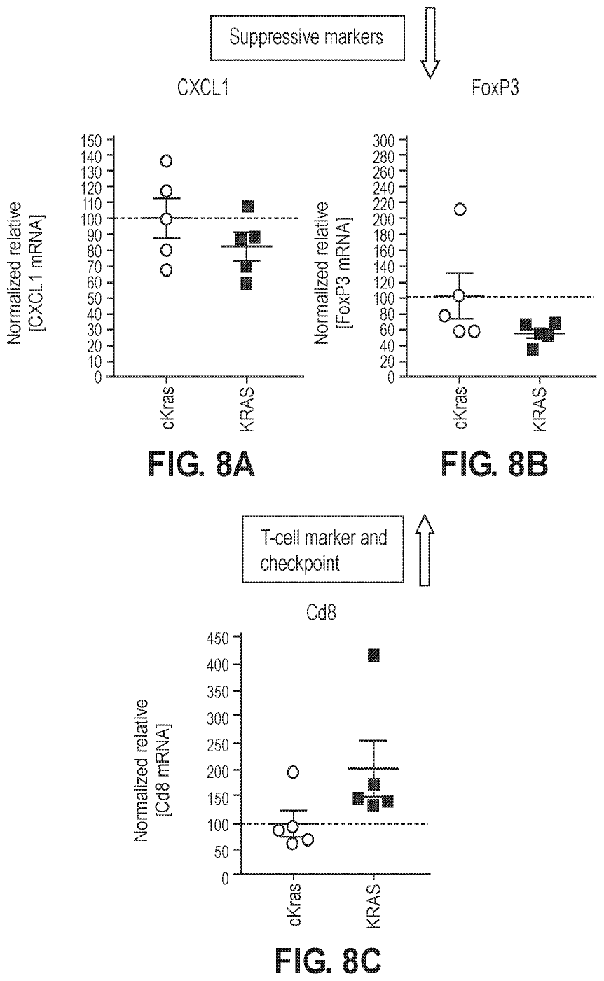

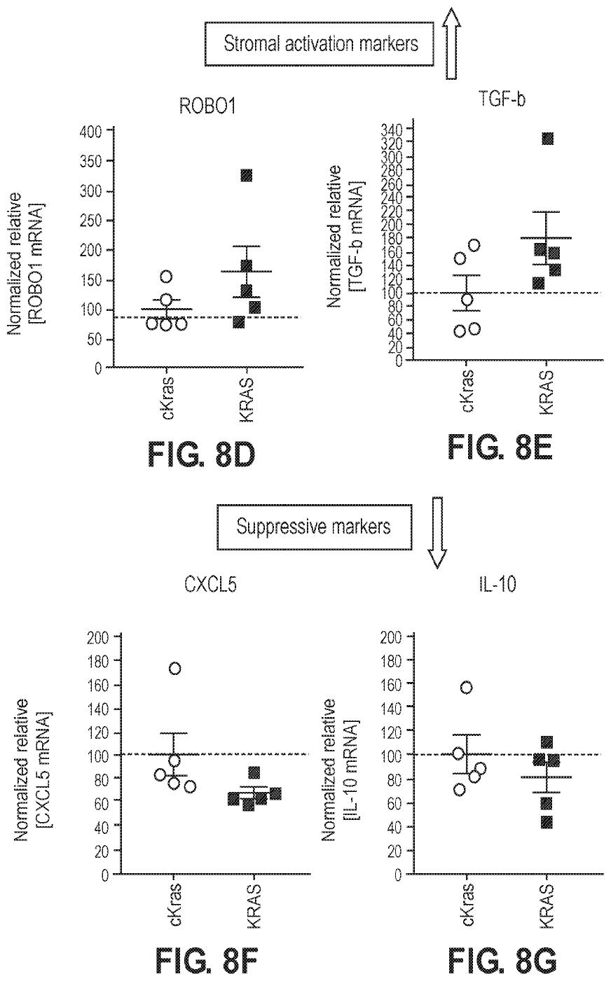

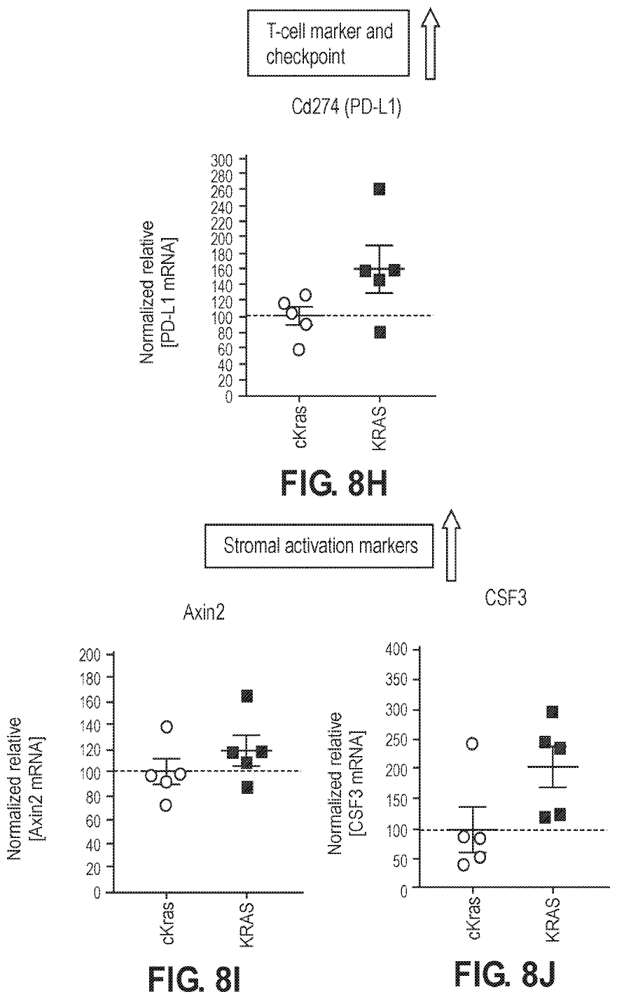

To see if continuous KRAS inhibition would lead to modulation of the tumor microenvironment, Pan02 tumors from the efficacy study described in Example 3 (FIG. 6) were collected 24 hours after the last dose and subjected to qPCR to measure mRNAs of immune cell markers (CD8, FoxP3), immune suppressive cytokines (CXCL1, CXCL5, and IL10), immune checkpoints (PD-L1) or stromal activation markers (TGF-0, Axin2, ROBO1, and CSF3). As shown in FIG. 6, KRAS DsiRNA treatment led to complete growth inhibition in these tumors. This in turn led to down-regulation of several key suppressive molecules (FoxP3, CXCL1, and CXCL5). See FIGS. 8A, 8B, and 8F. In addition, this also increased the levels of Cd8 mRNA and Cd274 (PD-L1) mRNA. See FIGS. 8C and 8H. However, all the stromal activation markers seemed to be slightly increased upon KRAS inhibition. See FIGS. 8D, 8E, 8I, and 8J. This data suggests that the continuous KRAS inhibition modulates the suppressive tumor microenvironment markers to favor T-cell infiltration but did not alter stromal activation.

### Example 5: MEKi/KRAS Treatment Modulates Tumor Microenvironment to Favor T-Cell Infiltration

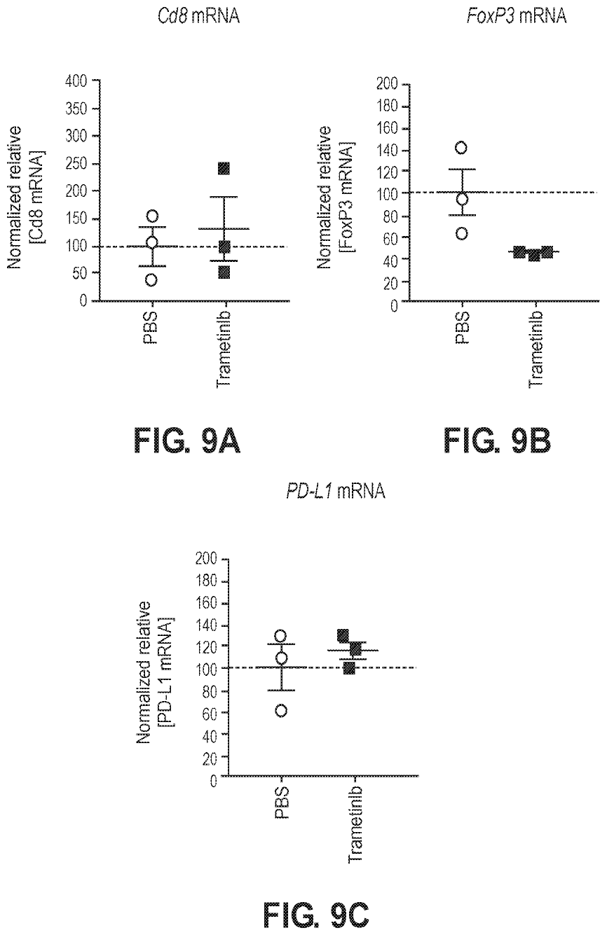

An FDA-approved MEK inhibitor, trametinib, is demonstrated to inhibit the MAPK pathway. To see how well the MEKi mediated inhibition alone modulates the tumor microenvironment, Pan02 tumors were implanted in C57BL/6 mice. At day 6, when tumors reached a size of about 200 mm³, they were treated with trametinib at 3 mpk for 3 days, i.e., at days 6, 7, and 8 post-tumor implantation (qdx3, 3 mpk). 24 hours after the last dose, i.e. day 9 post-tumor implantation, tumors were collected and analyzed for immune cell markers and other related markers (CD8, FoxP3, PD-L1 etc). FoxP3 mRNA levels were down-regulated with MEK inhibition (see FIG. 9B), however the Cd8 and Cd274 (PD-L1) mRNA levels were unchanged. See FIGS. 9A and 9C.

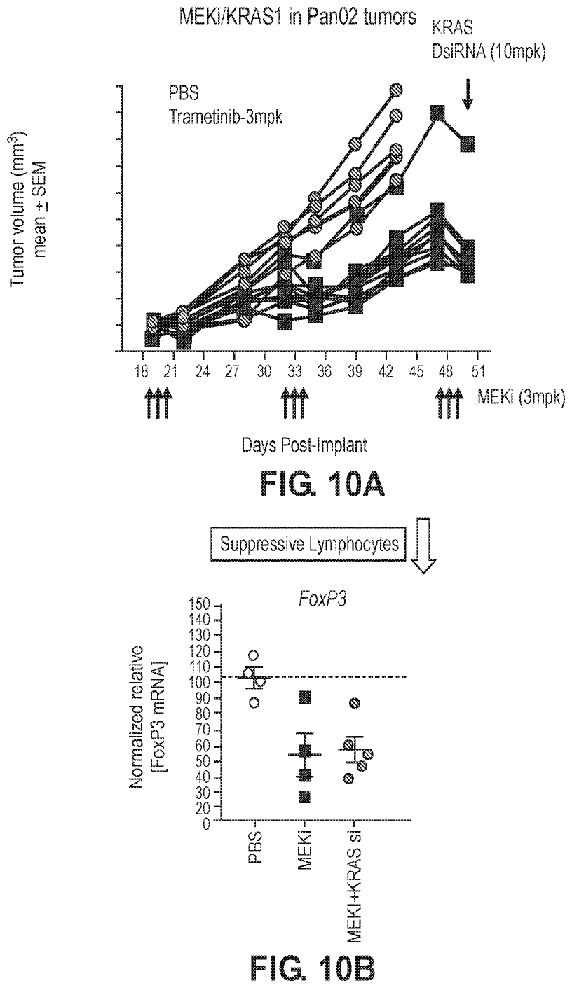

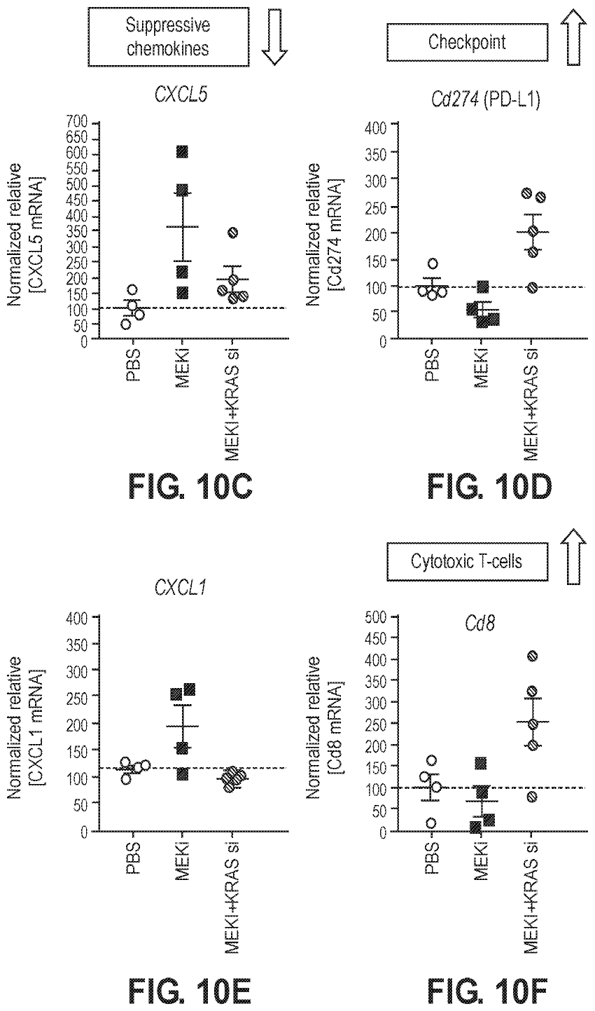

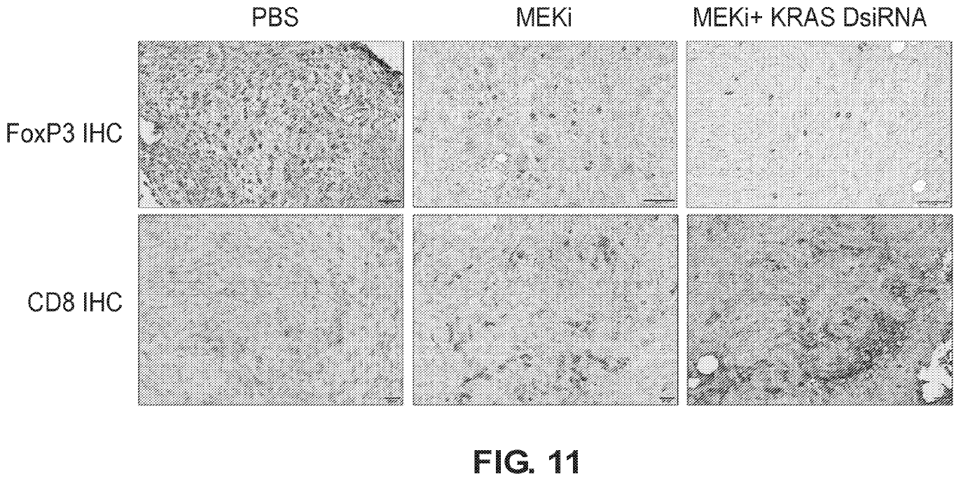

To see if continuous MEKi treatment would enhance CD8 T-cell infiltration, in another study, Pan02 tumors were treated with MEKi multiple times. After 3 cycles of treatment, 5 out of 10 mice that had the last MEKi treatment were treated additionally with KRAS1 at 10 mpk. See FIG. 10A. Tumors were collected before and after KRAS/LNP treatment and analyzed for T-cell markers (CD8, FoxP3), chemokine markers (CXCL1, CXCL5) and checkpoint (PD-L1). After 3 cycles of MEKi treatment, the mRNA levels of CXCL1 and CXCL5 were increased. However, a single KRAS/LNP treatment after the MEKi treatments reduced the mRNA levels of CXCL1 and CXCL5 to the background levels. See FIGS. 10C and 10E. KRAS/LNP treatment also increased the mRNA levels of Cd8 and Cd274 (PD-L1) that were decreased by the MEKi treatments. See FIGS. 10D and 10F. FoxP3 mRNA levels however, were decreased after MEKi and KRAS/LNP treatments. See FIG. 10B. These mRNA data suggest that MEKi alone is unable to reduce the suppressive chemokines/molecules to a level that can favor T-cell infiltration, whereas a single KRAS/LNP treatment effectively decreased many of the suppressive molecules and increased the levels of CD8 and PD-L1. This was demonstrated by FoxP3 and CD8 immunohistochemistry stained slides as well. See FIG. 11.

### Example 6: Direct Targeting of KRAS Evokes MEKi (Trametinib) and Gemcitabine Mediated Resistance in KRAS G12D Mutation Pancreatic Cancer

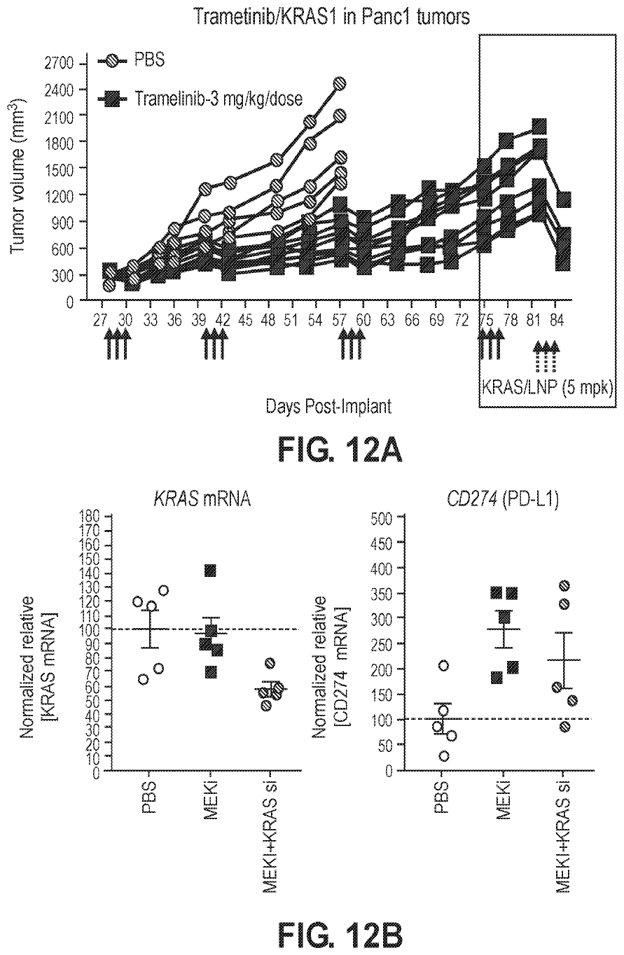

To see how trametinib performed in human PDAC, Panc1 tumors were implanted as described above and treated with trametinib (3 mg/kg/dose) as shown in FIG. 12A. Tumor measurements were taken throughout the entire study period to monitor the tumor growth. When the tumors stopped responding to trametinib treatment (considered that the tumors become resistant to trametinib), the tumors were then treated with KRAS/LNP at 5 mpk (qdx3). Tumors were collected before and after KRAS/LNP treatments for mRNA analysis. Interestingly, the trametinib-resistant Panc1 tumors responded to KRAS/LNP treatment and regressed. See FIG. 12A. KRAS1 treated tumors demonstrated about 40-50% KRAS knockdown after treatment compared to resistant tumors that didn't have KRAS/LNP treatment, suggesting that even when these tumors are resistant to targeted agents, they are still sensitive to KRAS DsiRNA. Interestingly, Cd274 (PD-L1) mRNA levels were increased in tumors that had MEKi or MEKi+KRAS DsiRNA treatments. See FIG. 12B.

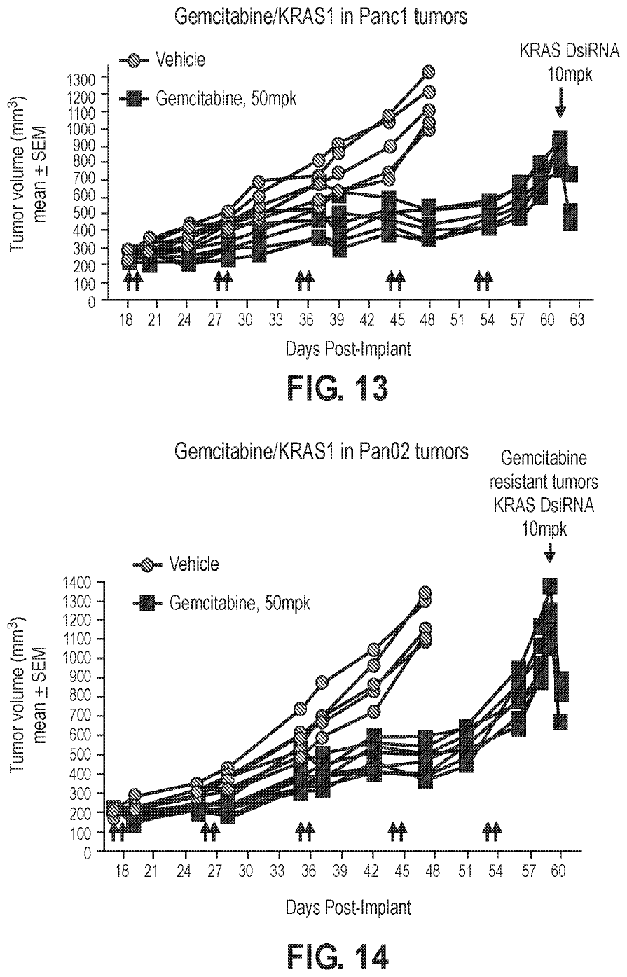

Similarly, in another study, Panc1 tumors were grown as described and treated with the current standard of care gemcitabine (50 mpk). Although the tumors responded nicely at the beginning, they became resistant after several rounds of treatment. See FIG. 13. Again, when these resistant tumors were treated with KRAS/LNP (10 mpk) as described above and as shown in FIG. 13, the resistant tumors responded to KRAS1 just like the trametinib resistant tumors responded to KRAS/LNP, suggesting that these tumors that become resistant to either targeted agent or chemotherapeutic agent, are still sensitive to KRAS/LNP.

A similar study was repeated in Pan02 tumors as well. Pan02 tumors were continuously treated with gemcitabine until they became resistant, and the resistant Pan02 tumors were then treated with KRAS1. See FIG. 14. Similar results were observed for both Panel tumors and Pan02 tumors. The gemcitabine-resistant Pan02 tumors responded well to KRAS/LNP and regressed. In this case, tumors were collected and analyzed for the mRNA markers that contribute to modulation of the tumor microenvironment and stromal activation.

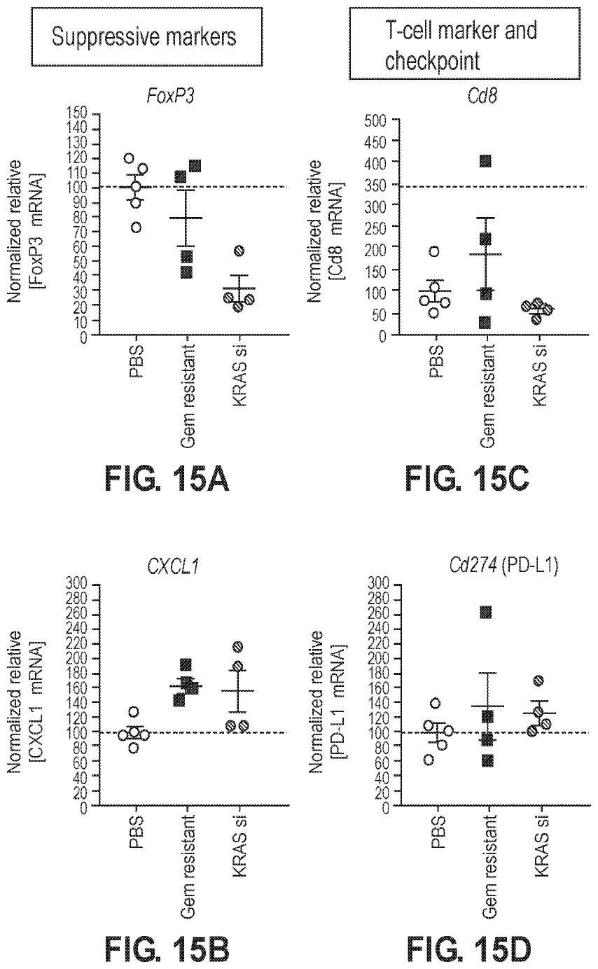

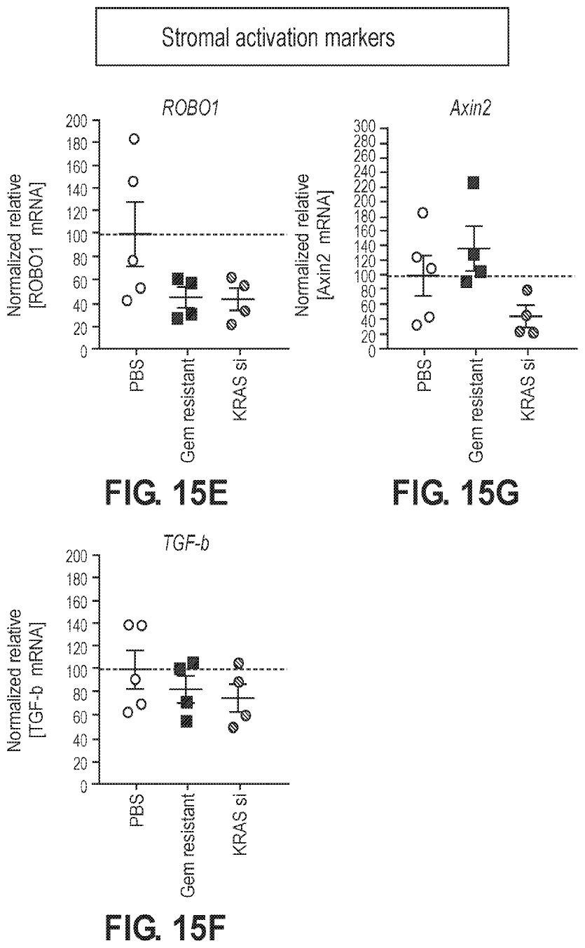

When the tumors were treated with gemcitabine until they became resistant, the CXCL1 mRNA levels increased. These levels were not brought down to baseline with a single KRAS1 treatment. See FIG. 15B. Gemcitabine treatment followed by KRAS/LNP treatment did not alter the mRNA levels of Cd8 and Cd274 (PD-L1). See FIGS. 15C and 15D. However, the Gemcitabine treatment±KRAS/LNP treatment seemed to lower some of the stromal activation markers (Axin2, ROBO1 and TGF-β). See FIGS. 15E-G). This suggests that gemcitabine treatment could be used to reduce the stromal activation but may not be good enough to bring down the suppressive immune cell markers in the tumor microenvironment.

### Example 7: Single Agent TGF-β Inhibitor or CSF1 Antibodies to Inactivate Stromal Markers

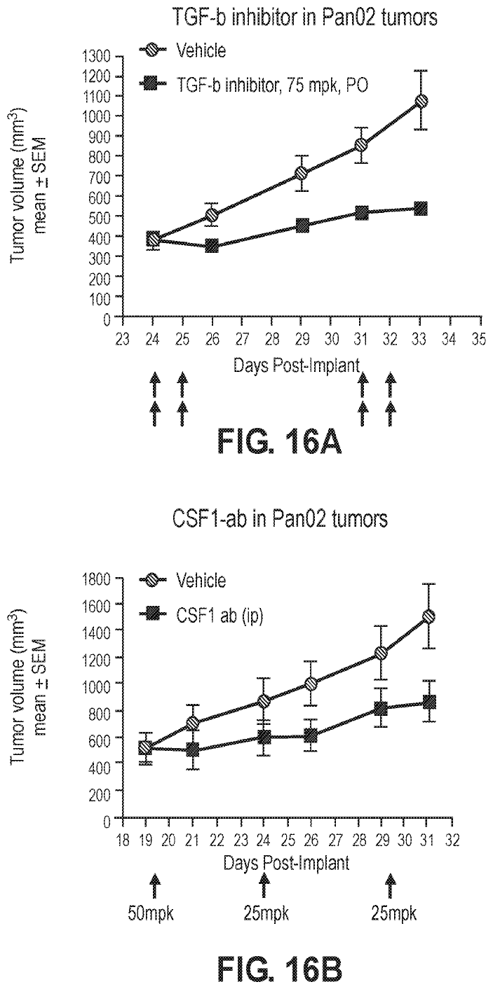

It may be desirable to bring down many of the suppressive molecules in the tumor microenvironment to increase CD8 T-cell infiltration in these Pan02 tumors. It is also evident that the inactivation of the stromal compartment may be equally important to keep up the effective T-cells in the tumor microenvironment, as stromal components play a role in promoting tumor growth and invasion. KRAS inhibition (up to about 40%) seemed to bring many suppressive molecules and increase CD8 T-cells, but did not seem to alter the stromal compartment. Since TGF-β and Wnt signaling pathways are implicated in activating stromal compartment, inhibitors that down-regulate one of these pathways were evaluated in these tumors. A TGF-β inhibitor (galunisertib) or CSF1 antibody (that is reported to reduce the macrophage accumulation and stromal content around PDACs) were used in studies to check the hypothesis. In one study, Pan02 tumors were either treated orally with TGF-β inhibitor at 75 mpk (BID×2/cycle) or Vehicle for 2 weeks, and the tumor growth was monitored throughout the study period. See FIG. 16A. In another study, Pan02 tumors were treated intraperitoneally with CSF1 antibodies (q5d, with a first dose at 50 mpk and consequent doses at 25 mpk), and tumor growth was monitored over time. See FIG. 16B. In both cases, a tumor growth inhibition of about 50% was seen. See FIGS. 16A-B. Tumors at the end of the study were also collected and analyzed for stromal activation markers (ROBO1, TGF-β, Axin2, etc.).

### Example 8: Combination of KRAS Inhibition Together with Drugs that Inactivate Stromal Activation

To incorporate the knowledge obtained from those single-agent treatments, a combination study may be designed and carried out. A drug that brings down the immunosuppressive molecules (a KRAS nucleic acid inhibitor molecule) may be combined with a drug that reduces the stromal activation (e.g., a TGF-β inhibitor or a CSF1 inhibitor) and a drug that relieves the checkpoint blockade. Pan02 tumors may be implanted and treated with KRAS/LNP, TGF-β inhibitor, and checkpoint inhibitors as described.

Unless otherwise indicated, all numbers used in the specification and claims are to be understood as being modified in all instances by the term “about,” whether or not so stated. It should also be understood that the precise numerical values used in the specification and claims form additional embodiments of the disclosure, as do all ranges and subranges within any specified endpoints. In addition, it will be noted that where steps are disclosed, the steps need not be performed in that order unless explicitly stated.

Other embodiments will be apparent to those skilled in the art from consideration of the specification and practice of the disclosure.

1. A method of treating a KRAS-associated cancer in a subject, comprising administering to the subject:
a therapeutically effective amount of a KRAS nucleic acid inhibitor molecule; and
a therapeutically effective amount of an MEK inhibitor.

2. The method of claim 1, wherein the MEK inhibitor is trametinib.

3. The method of claim 1, wherein the KRAS-associated cancer is resistant to treatment with the MEK inhibitor prior to administration of the KRAS nucleic acid inhibitor molecule.

4. A method of potentiating a therapeutic effect of an immunotherapeutic agent against a KRAS-associated cancer, comprising administering to a subject having the KRAS-associated cancer a KRAS nucleic acid inhibitor molecule in an amount sufficient to potentiate the therapeutic effect of the immunotherapeutic agent against the cancer.

5. The method of claim 4, wherein prior to administering the KRAS nucleic acid inhibitor molecule, the KRAS-associated cancer is associated with a non-T cell inflamed phenotype that is resistant to immunotherapy and wherein administering the KRAS nucleic acid inhibitor molecule converts the non-T cell inflamed phenotype into a T cell-inflamed phenotype that is responsive to an immunotherapeutic agent.

6. The method of claim 1, further comprising administering an agent that reduces stromal markers in the tumor microenvironment.

7. The method of claim 6, wherein the agent that reduces stromal markers in the tumor microenvironment is a TGF-β inhibitor or a CSF1 inhibitor.

8. A method of treating a KRAS-associated cancer in a subject, comprising administering to the subject:
a therapeutically effective amount of a KRAS nucleic acid inhibitor molecule, and a therapeutically effective amount of an immunotherapeutic agent.

9. The method of claim 4, wherein the immunotherapeutic agent is an antagonist of an inhibitory immune checkpoint molecule or an agonist of a co-stimulatory checkpoint molecule.

10. The method of claim 9, wherein the immunotherapeutic agent is an antagonist of an inhibitory check point, and the inhibitory check point is PD-1 or PD-L1.

11. The method of claim 9, wherein the antagonist of the inhibitory immune checkpoint molecule or the agonist of the co-stimulatory checkpoint molecule is a monoclonal antibody.

12. The method of claim 1, wherein the KRAS-associated cancer is pancreatic cancer.

13. The method of claim 1, wherein the KRAS nucleic acid inhibitor molecule is a double stranded RNAi inhibitor molecule comprising a sense stand and an antisense strand and a region of complementarity between the sense strand and the antisense strand of about 15-45 base pairs.

14. The method of claim 13, wherein the sense strand is 25-40 nucleotides and contains a stem and a loop, the antisense strand is 18-24 nucleotides and optionally comprises a single-stranded overhang of 1-2 nucleotides at its 3′-terminus, wherein the sense strand and antisense strand form a duplex region of 18-24 base pairs.

15. The method of claim 13, wherein the region of complementarity between the sense strand and the antisense strand is 21-26 nucleotides, wherein the sense strand is 21-26 nucleotides in length and wherein the antisense strand is 23-38 nucleotides in length and includes a single-stranded overhang of 1-2 nucleotides at its 3′-terminus.

16. The method of claim 15, wherein the antisense strand further comprises a single-stranded overhang of 1-5 nucleotides at its 5′-terminus.

17. The method of claim 13, wherein:
a) the sense strand is 26-36 nucleotides and contains a stem and a tetraloop, and the antisense strand is 18-24 nucleotides, wherein the sense strand and antisense strand form a duplex region of 18-24 nucleotides;
b) the sense strand is 34-36 nucleotides and contains a stem and a tetraloop, and the antisense strand is 18-24 nucleotides, wherein the sense strand and antisense strand form a duplex region of 18-24 nucleotides;
c) the sense strand is 34-36 nucleotides and contains a stem and a tetraloop, and the antisense strand is 18-24 nucleotides, wherein the sense strand and antisense strand form a duplex region of 18-24 nucleotides; or
d) the sense strand is 25-35 nucleotides and contains a stem and a triloop, and the antisense strand is 18-24 nucleotides, wherein the sense strand and antisense strand form a duplex region of 18-24 nucleotides.

18. The method of claim 13, wherein the region of complementarity between the sense strand and the antisense strand is 19 nucleotides, wherein the sense strand is 21 nucleotides in length and includes a single-stranded overhang of 2 nucleotides at its 3′-terminus and wherein the antisense strand is 21 nucleotides in length and includes a single-stranded overhang of 2 nucleotides at its 3′-terminus.

19. The method of claim 13, wherein the region of complementarity between the sense strand and the antisense strand is 21 nucleotides, wherein the sense strand is 21 nucleotides in length and wherein the antisense strand is 23 nucleotides in length and includes a single-stranded overhang of 2 nucleotides at its 3′-terminus.

20. The method or composition of claim 1, wherein the KRAS nucleic acid inhibitor molecule is formulated with a lipid nanoparticle.

21. The method of claim 20, wherein the lipid nanoparticle comprises a cationic lipid and a pegylated lipid.

22. The method of claim 13, wherein the sense strand comprises or consists of the sequence of SEQ ID NO: 13.

23. The method of claim 13, wherein the antisense strand comprises or consists of the sequence of SEQ ID NO: 14 or SEQ ID NO: 18.

24. The method of claim 14, wherein the sense strand comprises or consists of the sequence of one of SEQ ID NO: 15.

25. The method of claim 14, wherein the antisense sense strand comprises or consists of the sequence of one of SEQ ID NO: 16 or 19.

26. The method of claim 14, wherein:
(a) the sense strand comprises or consists of the sequence of SEQ ID NO: 3 and the antisense sense strand comprises or consists of the sequence of SEQ ID NO: 4;
(b) the sense strand comprises or consists of the sequence of SEQ ID NO: 1 and the antisense sense strand comprises or consists of the sequence of SEQ ID NO: 2; or
(c) the sense strand comprises or consists of the sequence of SEQ ID NO: 5 and the antisense sense strand comprises or consists of the sequence of SEQ ID NO: 6.

27. The method of claim 14, wherein:
(a) the sense strand comprises or consists of the sequence of SEQ ID NO: 7 and the antisense sense strand comprises or consists of the sequence of SEQ ID NO: 8;
(b) the sense strand comprises or consists of the sequence of SEQ ID NO: 9 and the antisense sense strand comprises or consists of the sequence of SEQ ID NO: 10;
(c) the sense strand comprises or consists of the sequence of SEQ ID NO: 11 and the antisense sense strand comprises or consists of the sequence of SEQ ID NO: 12;
(d) the sense strand comprises or consists of the sequence of SEQ ID NO: 7 and the antisense sense strand comprises or consists of the sequence of SEQ ID NO: 17;
(e) the sense strand comprises or consists of the sequence of SEQ ID NO: 13 and the antisense sense strand comprises or consists of the sequence of SEQ ID NO: 18;
(f) the sense strand comprises or consists of the sequence of SEQ ID NO: 15 and the antisense sense strand comprises or consists of the sequence of SEQ ID NO: 19;
(g) the sense strand comprises or consists of the sequence of SEQ ID NO: 13 and the antisense sense strand comprises or consists of the sequence of SEQ ID NO: 14; or
(h) the sense strand comprises or consists of the sequence of SEQ ID NO: 15 and the antisense sense strand comprises or consists of the sequence of SEQ ID NO: 16.


SEQUENCE LISTING (55 sequences)

SEQ ID NO: 1 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
ggccugcugaaaaugacugaauata

SEQ ID NO: 2 - RNA, 27 nt, Artificial Sequence
  source: /note=“Description of Artificial Sequence Synthetic oligonucleotide”
uauauucagucauuuucagcaggccuc

SEQ ID NO: 3 - RNA, 25 nt, Artificial Sequence
  source: /note=“Description of Artificial Sequence Synthetic oligonucleotide”
cuaaaucauuugaagauauucacca

SEQ ID NO: 4 - RNA, 27 nt, Artificial Sequence
  source: /note=“Description of Artificial Sequence Synthetic oligonucleotide”
uggugaauaucuucaaaugauuuagua

SEQ ID NO: 5 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
guauuugccauaaauaauacuaaat

SEQ ID NO: 6 - RNA, 27 nt, Artificial Sequence
  source: /note=“Description of Artificial Sequence Synthetic oligonucleotide”
auuuaguauuauuuauggcaaauacac

SEQ ID NO: 7 - RNA, 36 nt, Artificial Sequence
  source: /note=“Description of Artificial Sequence Synthetic oligonucleotide”
ggccugcugaaaaugacugagcagccgaaaggcugc

SEQ ID NO: 8 - RNA, 22 nt, Artificial Sequence
  source: /note=“Description of Artificial Sequence Synthetic oligonucleotide”
ucagucauuuucagcaggccuc

SEQ ID NO: 9 - RNA, 36 nt, Artificial Sequence
  source: /note=“Description of Artificial Sequence Synthetic oligonucleotide”
cuaaaucauuugaagauauugcagccgaaaggcugc

SEQ ID NO: 10 - RNA, 22 nt, Artificial Sequence
  source: /note=“Description of Artificial Sequence Synthetic oligonucleotide”
aauaucuucaaaugauuuagua

SEQ ID NO: 11 - RNA, 36 nt, Artificial Sequence
  source: /note=“Description of Artificial Sequence Synthetic oligonucleotide”
guauuugccauaaauaauacgcagccgaaaggcugc

SEQ ID NO: 12 - RNA, 22 nt, Artificial Sequence
  source: /note=“Description of Artificial Sequence Synthetic oligonucleotide”
guauuauuuauggcaaauacac

SEQ ID NO: 13 - RNA, 36 nt, Artificial Sequence
  source: /note=“Description of Artificial Sequence Synthetic oligonucleotide”
cuaaaucauuugaagauauagcagccgaaaggcugc

SEQ ID NO: 14 - RNA, 22 nt, Artificial Sequence
  source: /note=“Description of Artificial Sequence Synthetic oligonucleotide”
  modified_base (1)..(1): 4′-oxymethylphosphonate uracil
nauaucuucaaaugauuuaggg

SEQ ID NO: 15 - RNA, 36 nt, Artificial Sequence
  source: /note=“Description of Artificial Sequence Synthetic oligonucleotide”
guauuugccauaaauaauaagcagccgaaaggcugc

SEQ ID NO: 16 - RNA, 22 nt, Artificial Sequence
  source: /note=“Description of Artificial Sequence Synthetic oligonucleotide”
  modified_base (1)..(1): 4′-oxymethylphosphonate uracil
nuauuauuuauggcaaauacgg

SEQ ID NO: 17 - RNA, 22 nt, Artificial Sequence
  source: /note=“Description of Artificial Sequence Synthetic oligonucleotide”
ucagucauuuucagcaggccgg

SEQ ID NO: 18 - RNA, 22 nt, Artificial Sequence
  source: /note=“Description of Artificial Sequence Synthetic oligonucleotide”
uauaucuucaaaugauuuaggg

SEQ ID NO: 19 - RNA, 22 nt, Artificial Sequence
  source: /note=“Description of Artificial Sequence Synthetic oligonucleotide”
uuauuauuuauggcaaauacgg

SEQ ID NO: 20 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
gauggagaaaccugucucuuggata

SEQ ID NO: 21 - RNA, 27 nt, Artificial Sequence
  source: /note=“Description of Artificial Sequence Synthetic oligonucleotide”
uauccaagagacagguuucuccaucaa

SEQ ID NO: 22 - RNA, 25 nt, Artificial Sequence
  source: /note=“Description of Artificial Sequence Synthetic oligonucleotide”
ucuuggauauucucgacacagcagg

SEQ ID NO: 23 - RNA, 27 nt, Artificial Sequence
  source: /note=“Description of Artificial Sequence Synthetic oligonucleotide”
ccugcugugucgagaauauccaagaga

SEQ ID NO: 24 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
gaguacagugcaaugagggaccagt

SEQ ID NO: 25 - RNA, 27 nt, Artificial Sequence
  source: /note=“Description of Artificial Sequence Synthetic oligonucleotide”
acuggucccucauugcacuguacuccu

SEQ ID NO: 26 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
uugccauaaauaauacuaaaucatt

SEQ ID NO: 27 - RNA, 27 nt, Artificial Sequence
  source: /note=“Description of Artificial Sequence Synthetic oligonucleotide”
aaugauuuaguauuauuuauggcaaau

SEQ ID NO: 28 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
cauaaagaaaagaugagcaaagatg

SEQ ID NO: 29 - RNA, 27 nt, Artificial Sequence
  source: /note=“Description of Artificial Sequence Synthetic oligonucleotide”
caucuuugcucaucuuuucuuuauguu

SEQ ID NO: 30 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
uacauuacacuaaauuauuagcatt

SEQ ID NO: 31 - RNA, 27 nt, Artificial Sequence
  source: /note=“Description of Artificial Sequence Synthetic oligonucleotide”
aaugcuaauaauuuaguguaauguaca

SEQ ID NO: 32 - RNA, 25 nt, Artificial Sequence
  source: /note=“Description of Artificial Sequence Synthetic oligonucleotide”
cuaaauuauuagcauuuguuuuagc

SEQ ID NO: 33 - RNA, 27 nt, Artificial Sequence
  source: /note=“Description of Artificial Sequence Synthetic oligonucleotide”
gcuaaaacaaaugcuaauaauuuagug

SEQ ID NO: 34 - RNA, 25 nt, Artificial Sequence
  source: /note=“Description of Artificial Sequence Synthetic oligonucleotide”
uagcauuuguuuuagcauuaccuaa

SEQ ID NO: 35 - RNA, 27 nt, Artificial Sequence
  source: /note=“Description of Artificial Sequence Synthetic oligonucleotide”
uuagguaaugcuaaaacaaaugcuaau

SEQ ID NO: 36 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
uauauuuacaugcuacuaaauuutt

SEQ ID NO: 37 - RNA, 27 nt, Artificial Sequence
  source: /note=“Description of Artificial Sequence Synthetic oligonucleotide”
aaaaauuuaguagcauguaaauauagc

SEQ ID NO: 38 - RNA, 36 nt, Artificial Sequence
  source: /note=“Description of Artificial Sequence Synthetic oligonucleotide”
gauggagaaaccugucucuugcagccgaaaggcugc

SEQ ID NO: 39 - RNA, 22 nt, Artificial Sequence
  source: /note=“Description of Artificial Sequence Synthetic oligonucleotide”
aagagacagguuucuccaucaa

SEQ ID NO: 40 - RNA, 36 nt, Artificial Sequence
  source: /note=“Description of Artificial Sequence Synthetic oligonucleotide”
ucuuggauauucucgacacagcagccgaaaggcugc

SEQ ID NO: 41 - RNA, 22 nt, Artificial Sequence
  source: /note=“Description of Artificial Sequence Synthetic oligonucleotide”
ugugucgagaauauccaagaga

SEQ ID NO: 42 - RNA, 36 nt, Artificial Sequence
  source: /note=“Description of Artificial Sequence Synthetic oligonucleotide”
gaguacagugcaaugagggagcagccgaaaggcugc

SEQ ID NO: 43 - RNA, 22 nt, Artificial Sequence
  source: /note=“Description of Artificial Sequence Synthetic oligonucleotide”
ucccucauugcacuguacuccu

SEQ ID NO: 44 - RNA, 36 nt, Artificial Sequence
  source: /note=“Description of Artificial Sequence Synthetic oligonucleotide”
uugccauaaauaauacuaaagcagccgaaaggcugc

SEQ ID NO: 45 - RNA, 22 nt, Artificial Sequence
  source: /note=“Description of Artificial Sequence Synthetic oligonucleotide”
uuuaguauuauuuauggcaaau

SEQ ID NO: 46 - RNA, 36 nt, Artificial Sequence
  source: /note=“Description of Artificial Sequence Synthetic oligonucleotide”
cauaaagaaaagaugagcaagcagccgaaaggcugc

SEQ ID NO: 47 - RNA, 22 nt, Artificial Sequence
  source: /note=“Description of Artificial Sequence Synthetic oligonucleotide”
uugcucaucuuuucuuuauguu

SEQ ID NO: 48 - RNA, 36 nt, Artificial Sequence
  source: /note=“Description of Artificial Sequence Synthetic oligonucleotide”
uacauuacacuaaauuauuagcagccgaaaggcugc

SEQ ID NO: 49 - RNA, 22 nt, Artificial Sequence
  source: /note=“Description of Artificial Sequence Synthetic oligonucleotide”
uaauaauuuaguguaauguaca

SEQ ID NO: 50 - RNA, 36 nt, Artificial Sequence
  source: /note=“Description of Artificial Sequence Synthetic oligonucleotide”
cuaaauuauuagcauuuguugcagccgaaaggcugc

SEQ ID NO: 51 - RNA, 22 nt, Artificial Sequence
  source: /note=“Description of Artificial Sequence Synthetic oligonucleotide”
aacaaaugcuaauaauuuagug

SEQ ID NO: 52 - RNA, 36 nt, Artificial Sequence
  source: /note=“Description of Artificial Sequence Synthetic oligonucleotide”
uagcauuuguuuuagcauuagcagccgaaaggcugc

SEQ ID NO: 53 - RNA, 22 nt, Artificial Sequence
  source: /note=“Description of Artificial Sequence Synthetic oligonucleotide”
uaaugcuaaaacaaaugcuaau

SEQ ID NO: 54 - RNA, 36 nt, Artificial Sequence
  source: /note=“Description of Artificial Sequence Synthetic oligonucleotide”
uauauuuacaugcuacuaaagcagccgaaaggcugc

SEQ ID NO: 55 - RNA, 22 nt, Artificial Sequence
  source: /note=“Description of Artificial Sequence Synthetic oligonucleotide”
uuuaguagcauguaaauauagc